(19) United States
(12) Patent Application Publication
(10) Pub. No.: US 2026/0037872 A1
(43) Pub. Date: Feb. 5, 2026

(54) System and Method for Feature-Based Machine Learning (ML) Model Prediction

(71) Applicant: Northeastern University, Boston, MA (US)

(72) Inventors: Raimond Winslow, Portland, ME (US); Qingchu Jin, Portland, ME (US)

(21) Appl. No.: 19/288,506

(22) Filed: Aug. 1, 2025

Related U.S. Application Data
(60) Provisional application No. 63/678,985, filed on Aug. 2, 2024.

(51) Int. Cl. G06N 20/00; G16H 50/30

(52) U.S. Cl. CPC G06N 20/00; G16H 50/30

(57) ABSTRACT

A computer-based system and corresponding method perform feature-based machine learning (ML) model prediction. The system uses an imputation method to produce posterior distributions of unprovided features of a set of retrospective features. The posterior distributions are produced based on the set of retrospective features and provided features of the set of retrospective features. The system employs an ML model to produce a threshold and a risk score distribution of a prediction of an event and selects at least one unprovided feature from a partial set of the unprovided features to improve predictive accuracy of the ML model iteratively. The system outputs a representation of the at least one unprovided feature selected toward approximating a full-feature-capacity (FFC) prediction with a partial set of the retrospective features. The system enables efficient feature acquisition for accurate ML model prediction.

### RELATED APPLICATION

This application claims the benefit of U.S. Provisional Application No. 63/678,985, filed on Aug. 2, 2024. The entire teachings of the above application are incorporated herein by reference.

### BACKGROUND

A machine learning (ML) model is a program that learns from data to make predictions or decisions. Input to the ML model includes features (variables). The ML model learns to map the features to a target output based on patterns the ML model identifies in training data.

### SUMMARY

According to an example embodiment, a computer-implemented method for feature-based machine learning (ML) model prediction comprises using an imputation method to produce posterior distributions of unprovided features of a set of retrospective features. The posterior distributions are produced based on the set of retrospective features and provided features of the set of retrospective features. The computer-implemented method further comprises producing, by an ML model, a threshold and a risk score distribution of a prediction of an event. The producing is based on the posterior distributions produced by the imputation method used and the provided features. The ML model is trained on the set of retrospective features. The computer-implemented method further comprises selecting at least one unprovided feature from a partial set of the unprovided features to improve predictive accuracy of the ML model iteratively. The selecting is based on the threshold and the risk score distribution of the prediction of the event. The computer-implemented method further comprises outputting a representation of the at least one unprovided feature selected toward approximating a full-feature-capacity (FFC) prediction with a partial set of the retrospective features. The FFC prediction is based on the set of retrospective features in its entirety. The representation output causes the at least one unprovided feature to be provided for a subsequent iteration. The partial set of the retrospective features includes the provided features supplemented by the at least one unprovided feature selected and provided at the subsequent iteration.

The event may be at least one event of a plurality of events. The computer-implemented method may further comprise applying at least one criterion to reduce the partial set of the unprovided features as a function of at least one characterization of the at least one event. A given event of the at least one event may be a time sensitive event.

The computer-implemented method may further comprise determining, based on the threshold and the risk score distribution, whether the provided features are sufficient for the ML model to approximate the FFC prediction. The computer-implemented method may further comprise performing the selecting of the at least one unprovided feature and the outputting of the representation responsive to determining that the provided features are not sufficient for approximating the FFC prediction. The computer-implemented method may further comprise outputting the prediction responsive to determining that the provided features are sufficient for approximating the FFC prediction.

The event may be a medical event for a patient for non-limiting example. The computer-implemented method may further comprise producing a decision based on the prediction output. The decision produced may influence triage of the patient to prevent the medical event.

The set of retrospective features may include clinical features of patients on a per-patient basis for non-limiting example. The event may be associated with a medical outcome of a patient. The risk score distribution of the prediction may represent certainty of the prediction in a presence of the unprovided features. The threshold may be learned by the ML model from the set of retrospective features in a training phase of the ML model.

The representation may indicate a respective feature importance ranking for each unprovided feature selected of the at least one unprovided feature selected. The respective feature importance ranking may indicate relative importance, among the at least one unprovided feature selected, toward improving the predictive accuracy of the ML model.

The using, producing, selecting, and outputting may be performed in a current iteration. The computer-implemented method may further comprise acquiring the at least one unprovided feature selected. The acquiring may be responsive to the outputting of the current iteration. The computer-implemented method may further comprise updating the provided features to include the at least one unprovided feature selected and acquired for use in the subsequent iteration.

The acquiring may include causing at least one device to perform at least one measurement to measure an unprovided feature of the at least one unprovided feature selected.

The computer-implemented method may further comprise employing the computer-implemented method in a computer-based tool for clinical evaluation of a patient for non-limiting example. The computer-implemented method may further comprise performing, by the computer-based tool, dynamic risk assessment of the patient based on the threshold and the risk score distribution of the prediction of the event. The event may be a medical outcome for the patient for non-limiting example.

The event may be a medical outcome for a patient for non-limiting example and the computer-implemented method may further comprise outputting an indication that represents at least one actionable component for preventing the medical outcome from occurring.

The ML model may be a supervised ML model.

According to another example embodiment, a computer-based system for feature-based machine learning (ML) model prediction comprises at least one processor and at least one memory. The at least one memory has encoded thereon a sequence of instructions which, when loaded and executed by the at least one processor, causes the computer-based system to use an imputation method to produce posterior distributions of unprovided features of a set of retrospective features. The posterior distributions produced may be based on the set of retrospective features and provided features of the set of retrospective features. The sequence of instructions, when loaded and executed by the at least one processor, further causes the computer-based system to employ an ML model to produce a threshold and a risk score distribution of a prediction of an event. The producing is based on the posterior distributions produced by the imputation method used and the provided features. The ML model is trained on the set of retrospective features. The sequence of instructions, when loaded and executed by the at least one processor, further causes the computer-based system to select at least one unprovided feature from a partial set of the unprovided features to improve predictive accuracy of the ML model iteratively. Selection of the at least one unprovided feature is based on the threshold and the risk score distribution of the prediction of the event. The sequence of instructions, when loaded and executed by the at least one processor, further causes the computer-based system to output a representation of the at least one unprovided feature selected toward approximating a full-feature-capacity (FFC) prediction with a partial set of the retrospective features. The FFC prediction is based on the set of retrospective features in its entirety. The representation output causes the at least one unprovided feature to be provided for a subsequent iteration. The partial set of the retrospective features includes the provided features supplemented by the at least one unprovided feature selected and provided at the subsequent iteration.

Alternative computer-based system embodiments parallel those disclosed above in connection with the example computer-implemented method embodiment.

According to another example embodiment, a non-transitory computer-readable medium having encoded thereon a sequence of instructions which, when loaded and executed by at least one processor, causes the at least one processor to use an imputation method to produce posterior distributions of unprovided features of a set of retrospective features. The posterior distributions produced are based on the set of retrospective features and provided features of the set of retrospective features. The sequence of instructions further causes the processor to employ an ML model to produce a threshold and a risk score distribution of a prediction of an event. The producing is based on the posterior distributions produced by the imputation method used and the provided features. The ML model is trained on the set of retrospective features. The sequence of instructions further causes the processor to select at least one unprovided feature from a partial set of the unprovided features to improve predictive accuracy of the ML model iteratively. Selection of the at least one unprovided feature is based on the threshold and the risk score distribution of the prediction of the event. The sequence of instructions further causes the processor to output a representation of the at least one unprovided feature selected toward approximating a full-feature-capacity (FFC) prediction with a partial set of the retrospective features. The FFC prediction is based on the set of retrospective features in its entirety. The representation output causes the at least one unprovided feature to be provided for a subsequent iteration. The partial set of the retrospective features includes the provided features supplemented by the at least one unprovided feature selected and provided at the subsequent iteration.

Alternative non-transitory computer-readable medium embodiments parallel those disclosed above in connection with the example computer-implemented method embodiment.

It should be understood that example embodiments disclosed herein can be implemented in the form of a method, apparatus, system, or non-transitory computer readable medium with program codes embodied thereon.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a block diagram of an example embodiment of computer-based system for feature-based machine learning (ML) model prediction.

FIG. 2 is a flow diagram of an example embodiment of computer-based system for feature-based ML model prediction.

FIG. 3 is a block diagram of an example embodiment of a Feature Sufficiency Analysis (FSA) system.

FIG. 4A is a graph of an example embodiment of Society of Thoracic Surgeons (STS) prolonged ventilation prediction.

FIG. 4B is a graph of an example embodiment for 10-year mortality prediction per number of patients and features.

FIGS. 5A-T are graphs of results that illustrate validation of the estimated posterior distribution.

FIG. 6A is a graph of an example embodiment of a comparison between FSA-based feature ranking with 4 widely-used ranking methods.

FIG. 6B is a table of an example embodiment of a pairwise spearman correlation between the different ranking methods of FIG. 6A.

FIG. 6C is a table that summarizes the five different feature ranking methods of FIGS. 6A and 6B.

FIGS. 7A and 7B are graphs of example embodiments of distributions of a minimum number of features needed to make a full-feature-capacity (FFC) prediction.

FIGS. 7C-H are boxplots that demonstrate an example embodiment of an evolution of risk score distribution with an increasing number of features available.

FIG. 8A is a graph of an example embodiment of cumulative feature sufficiency analysis for prolonged ventilation.

FIG. 8B is a graph of an example embodiment of cumulative feature sufficiency analysis for mortality prediction.

FIG. 9A is graph of an example embodiment of a percentage of FFC-predictable patients given time.

FIG. 9B is graph of an example embodiment of a percentage of FFC-predictable patients given monetary cost

FIG. 10 is a graph of an example embodiment of a cost analysis for the 10-year mortality.

FIG. 11A is a graph of ML model performance for subsets of features for prolonged ventilation.

FIG. 11B is a graph of ML model performance for subsets of features for mortality prediction.

FIGS. 12A-D are graphs of example embodiments of grouping patients into easy and hard groups.

FIGS. 13A and 13B are graphs of example embodiment of a hard-to-predict group.

FIGS. 13C and 13D are tables of example embodiments of high predictive uncertainty (PU) groups.

FIG. 14 is a block diagram of an example of the internal structure of a computer in which various embodiments of the present disclosure may be implemented.

### DETAILED DESCRIPTION

A description of example embodiments follows.

It should be understood that an imputation method disclosed herein is for non-limiting example and may be any type of imputation method. Further, it should be understood that an example embodiment disclosed herein that employs a machine learning (ML) is agnostic with regard to a type of the ML model.

Feature(s) may be referred to interchangeably herein as a variable(s). Available feature(s) may be referred to interchangeably herein as provided feature(s). Unavailable feature(s) may be referred to interchangeably herein as unprovided feature(s).

While an example embodiment disclosed herein may be described in the context of a particular field of use, such as healthcare, it should be understood that embodiments disclosed herein are not limited to a particular field of use and may be employed in any field that deals with missing data in any type of machine-learning (ML) based prediction problem and are not limited to prediction of future events in patients. Example embodiments disclosed herein may be used in any type of ML prediction or classification application.

Machine learning studies in the healthcare application have expanded significantly in recent years, including using images to classify cancer (Esteva A, Kuprel B, Novoa R A, Ko J, Swetter S M, Blau H M, Thrun S. Dermatologist-level classification of skin cancer with deep neural networks. nature. 2017; 542 (7639): 115-8),using longitudinal ICU data to predict septic shock (Liu R, Greenstein J L, Granite S J, Fackler J C, Bembea M M, Sarma S V, Winslow R L. Data-driven discovery of a novel sepsis pre-shock state predicts impending septic shock in the ICU. Scientific reports. 2019; 9(1): 6145), using waveform data to predict neurological outcome of cardiac arrest patients (Kim H B, Nguyen H T, Jin Q, Tamby S, Romer T G, Sung E, Liu R, Greenstein J L, Suarez J I, Storm C. Computational signatures for post-cardiac arrest trajectory prediction: Importance of early physiological time series. Anaesthesia Critical Care & Pain Medicine. 2022; 41(1): 101015), using clinical code to predict the pancreatic cancer risk (Placido D, Yuan B, Hjaltelin J X, Zheng C, Hauc A D, Chmura P J, Yuan C, Kim J, Umeton R, Antell G. A deep learning algorithm to predict risk of pancreatic cancer from disease trajectories. Nature medicine. 2023; 29(5): 1113-22), using lab result data to diagnose ovarian cancer (Cai G, Huang F, Gao Y, Li X, Chi J, Xie J, Zhou L, Feng Y, Huang H, Deng T. Artificial intelligence-based models enabling accurate diagnosis of ovarian cancer using laboratory tests in China: a multicentre, retrospective cohort study. The Lancet Digital Health. 2024) and use multimodal data to predict severity of COVID-19 patients (Lassau N, Ammari S, Chouzenoux E, Gortais H, Herent P, Devilder M, Soliman S, Meyrignac O, Talabard M-P, Lamarque J-P. Integrating deep learning CT-scan model, biological and clinical variables to predict severity of COVID-19 patients. Nature communications. 2021; 12(1):1-11).

Enhancing the performance of AI often involves integrating a wide array of features. For example, models that utilize multimodal data have been shown to outperform a single modality (Kim H B, Nguyen H T, Jin Q, Tamby S, Romer T G, Sung E, Liu R, Greenstein J L, Suarez J I, Storm C. Computational signatures for post-cardiac arrest trajectory prediction: Importance of early physiological time series. Anaesthesia Critical Care & Pain Medicine. 2022; 41(1):101015). Soenksen et al. systematically demonstrated that multimodal models has a consistently improvement of performance across various healthcare applications, from lung lesion prediction to 48-hour mortality (Soenksen L R, Ma Y, Zeng C, Boussioux L, Villalobos Carballo K, Na L, Wiberg H M, Li M L, Fuentes I, Bertsimas D. Integrated multimodal artificial intelligence framework for healthcare applications. NPJ digital medicine. 2022; 5(1):149). However, while an increased number of features can boost performance, it also increases the operational costs. The cost contains not only the monetary aspect, but the time required for data acquisition, which is particularly critical in a time-sensitive clinical decision-making scenario such as the surgical triage of the non-elective surgery (Kluger Y, Ben-Ishay O, Sartelli M, Ansaloni L, Abbas A E, Agresta F, Biffl W L, Baiocchi L, Bala M, Catena F. World society of emergency surgery study group initiative on Timing of Acute Care Surgery classification (TACS). World Journal of Emergency Surgery. 2013; 8:1-6). Delaying the triage decision for waiting for the full feature list can potentially lead to an increase of risk of mortality and morbidity (McIsaac D I, Abdulla K, Yang H, Sundaresan S, Doering P, Vaswani S G, Thavorn K, Forster A J. Association of delay of urgent or emergency surgery with mortality and use of healthcare resources: a propensity score-matched observational cohort study. Cmaj. 2017; 189(27):E905-E12).

Recently, some contributions have been made to address the cost of the feature acquisition. Erion et al. developed a cost-aware framework (coAI) to create a collection of models, where each model is within a subset of features balancing the prediction performance and the feature cost based on the SHAP value (Erion G, Janizek J D, Hudelson C, Utarnachitt R B, McCoy A M, Sayre M R, White N J, Lee S-I. A cost-aware framework for the development of AI models for healthcare applications. Nature Biomedical Engineering. 2022; 6(12):1384-98). Clinicians can decide to choose the model based on the tolerated cost. Cost efficient gradient boosting (CEGB), a cost-aware prediction with decision trees (Peter S, Diego F, Hamprecht F A, Nadler B. Cost efficient gradient boosting. Advances in neural information processing systems. 2017; 30). These two methods provide a strategy to select subset of features for prediction based on the cost.

Beyond the cost of data acquisition, we believe that the necessity of features for prediction is inherently specific to each patient. That is to say, the number of features needed to achieve an accurate diagnosis may vary: some patients may need a broader array of features (multimodal data), while other may need fewer. Likewise, when considering a ML model, a full feature list may be useful for making a confident prediction, whereas a handful of features are enough for others. To address this, a hypothesis disclosed herein is that not every patient requires the entire feature list to enable the model to predict with its full capacity. Here ‘full model capacity (FMC) prediction’ means that the ML model will provide the same prediction with a partial set of features as it would with the full set of features. The minimal necessary features to make full model capacity prediction may be unique for each patient.

A computational framework, identified as a Feature Sufficiency Analysis (FSA) system, is disclosed herein and ascertains whether a subset of features is sufficient for the ML model to deliver a prediction with full model capacity. This framework may be based on Bayesian approach alongside with uncertainty analysis to determine the impact of missing features on the ML model's predictive accuracy. Thus, this tool is model-agnostic, ensuring compatibility across various ML models, and it generates tailored inference to each patient.

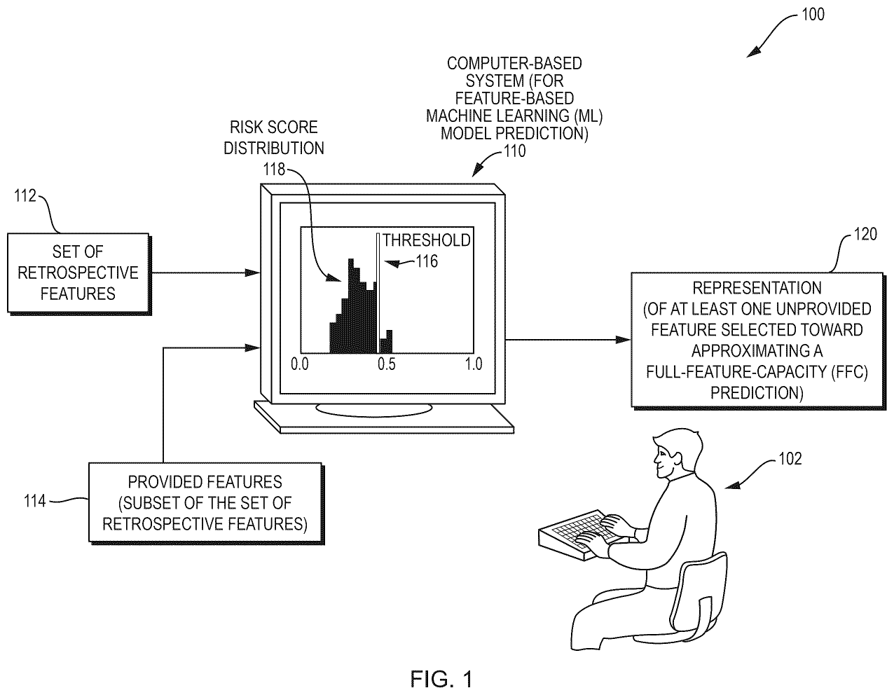

An example embodiment of a Feature Sufficiency Analysis (FSA) disclosed herein may be configured to evaluate whether a full model capacity (FMC) prediction is feasible with a partially observed set of features, such as patient features for non-limiting example. Although a case study disclosed herein utilized the STS ACSD cohort and random forest baseline model for demonstration, it should be understood that an example embodiment of a FSA system disclosed herein can be applied to any predictive task and ML method. As disclosed below, an example embodiment of an FSA system may employ a historical database of a set of retrospective features along with an observed feature subset (provide features, available features) to infer a posterior distribution of unobserved (unprovided, unavailable) features. This combined information-observed feature subset and inferred posterior distribution of unobserved features—may then propagated through a baseline (trained) ML model to yield a distribution of risk score. The risk score distribution may be assessed against the predetermined the threshold. If the entirety of the distribution surpasses this threshold, it indicates that the current feature set suffices for the ML model to make FMC prediction despite the uncertainty of unobserved features. In contrast, if the risk score distribution intersects with the threshold, this uncertainty indicates that additional features should be obtained for accurate prediction. An example embodiment of such an FSA system is disclosed below with regard to FIG. 1.

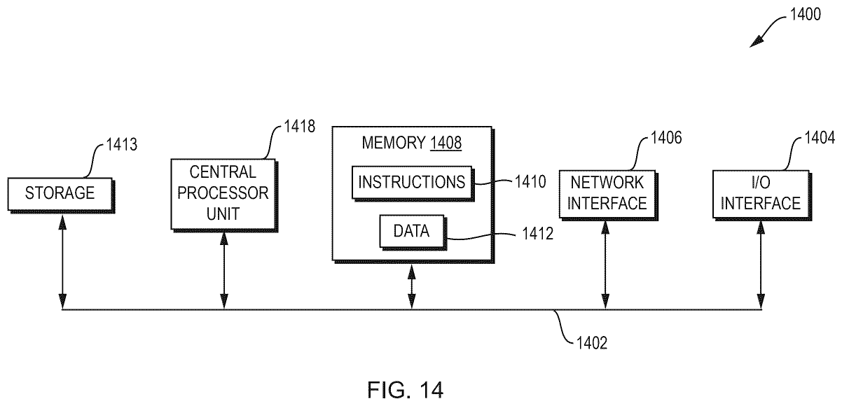

FIG. 1 is a block diagram 100 of a computer-based system 110 for feature-based machine learning (ML) model prediction. The computer-based system 110 may be referred to interchangeably herein as an FSA system. The computer-based system 110 may comprise at least one processor (not shown) and at least one memory (not shown) with computer code instructions stored thereon, such as disclosed further below in reference to FIG. 14 for non-limiting example. Continuing with reference to FIG. 1, the at least one memory has encoded thereon a sequence of instructions (not shown) which, when loaded and executed by the at least one processor, causes the computer-based system 110 to use an imputation method (not shown) to produce posterior distributions (not shown) of unprovided features (not shown) of a set of retrospective features 112, the posterior distributions produced based on the set of retrospective features 112 and provided features 114 of the set of retrospective features 112. The sequence of instructions, when loaded and executed by the at least one processor, may further cause the computer-based system to employ an ML model (not shown) to produce a threshold 116 and a risk score distribution 118 of a prediction (not shown) of an event (not shown).

Producing of the threshold 116 and risk score distribution 118 may be based on the posterior distributions produced by the imputation method used and the provided features 114. The ML model may be trained on the set of retrospective features 112. The sequence of instructions, when loaded and executed by the at least one processor, may further cause the computer-based system 110 to select at least one unprovided feature (not shown) from a partial set (not shown) of the unprovided features to improve predictive accuracy of the ML model iteratively. Selection of the at least one unprovided feature may be based on the threshold 116 and the risk score distribution 118 of the prediction of the event. The sequence of instructions, when loaded and executed by the at least one processor, may further cause the computer-based system 110 to output a representation 120 of the at least one unprovided feature selected toward approximating a full-feature-capacity (FFC) prediction (not shown) with a partial set (not shown) of the set of retrospective features 112. The FFC prediction may be based on the set of retrospective features 112 in its entirety. The representation 120 output may cause the at least one unprovided feature to be provided for a subsequent iteration. The partial set of the retrospective features may include the provided features 114 supplemented by the at least one unprovided feature selected and provided at the subsequent iteration.

The event may be at least one event of a plurality of events. The sequence of instructions, when loaded and executed by the at least one processor, may further cause the computer-based system to apply at least one criterion to reduce the partial set of the unprovided features as a function of at least one characterization of the at least one event. A given event of the at least one event may be a time sensitive event.

For non-limiting example, the event may be a medical event for a patient. The sequence of instructions, when loaded and executed by the at least one processor, may further cause the computer-based system 110 to determine, based on the threshold 116 and the risk score distribution 118, whether the provided features 114 are sufficient for the ML model to approximate the FFC prediction. The sequence of instructions, when loaded and executed by the at least one processor, may further cause the computer-based system 110 to perform selection of the at least one unprovided feature and to output the representation 120 responsive to determining that the provided features 114 are not sufficient for approximating the FFC prediction. The sequence of instructions, when loaded and executed by the at least one processor, may further cause the computer-based system 110 to output the prediction responsive to determining that the provided features 114 are sufficient for approximating the FFC prediction and to produce a decision based on the prediction output.

According to a non-limiting example, the computer-based system 112 may provide a highest marginal clinical utility. The decision produced may influence triage of the patient. For example, a user 102 of the computer-based system 110 may be a clinician for non-limiting example. For non-limiting example, a decision produced may to admit the patient for surgery in a given timeframe based on a predication of a time evolving mortality risk. Such a decision may be provided with a confidence level, represented by a risk score, and may save the patient's life as the user 102, as the clinician, may otherwise have waited too long to decide to admit the patient for surgery by conducting time consuming tests that do not increase confidence in a clinician's decision. According to a non-limiting example embodiment, the computer-based system 110 may output a list of at least one unprovided (missing, unavailable) feature that causes the user 102, as the clinician, to obtain the at least one unprovided feature toward providing same as at least one provided feature that is input to the computer-based system 100. The list may inform the user 102 of the missing data that, if obtained, would have the highest likelihood to eliminate the uncertainty of the decision produced.

For non-limiting example, the set of retrospective features 112 may include clinical features of patients on a per-patient basis. The event may be associated with a medical outcome of a patient. The risk score distribution 118 of the prediction may represent certainty of the prediction in a presence of the unprovided features. The threshold 116 may be learned by the ML model from the set of retrospective features 112 in a training phase of the ML model.

The representation 120 may indicate a respective feature importance ranking for each unprovided feature selected of the at least one unprovided feature selected. The respective feature importance ranking may indicate relative importance, among the at least one unprovided feature selected, toward improving the predictive accuracy of the ML model.

The sequence of instructions, when loaded and executed by the at least one processor, may further cause the computer-based system to acquire the at least one unprovided feature selected responsive to outputting the representation 120 in a current iteration and to update the provided features 114 to include the at least one unprovided feature selected and acquired for use in the subsequent iteration. Acquiring the at least one unprovided feature selected may include causing at least one device (not shown) to perform at least one measurement to measure an unprovided feature of the at least one unprovided feature selected.

For non-limiting example, the computer-based system 110 may be a tool for clinical evaluation of a patient and the sequence of instructions, when loaded and executed by the at least one processor, may further cause the computer-based system to perform dynamic risk assessment of the patient based on the threshold and the risk score distribution of the prediction of the event. The event may be a medical outcome for the patient and the sequence of instructions, when loaded and executed by the at least one processor, may further cause the computer-based system to output an indication that represents at least one actionable component for preventing the medical outcome from occurring.

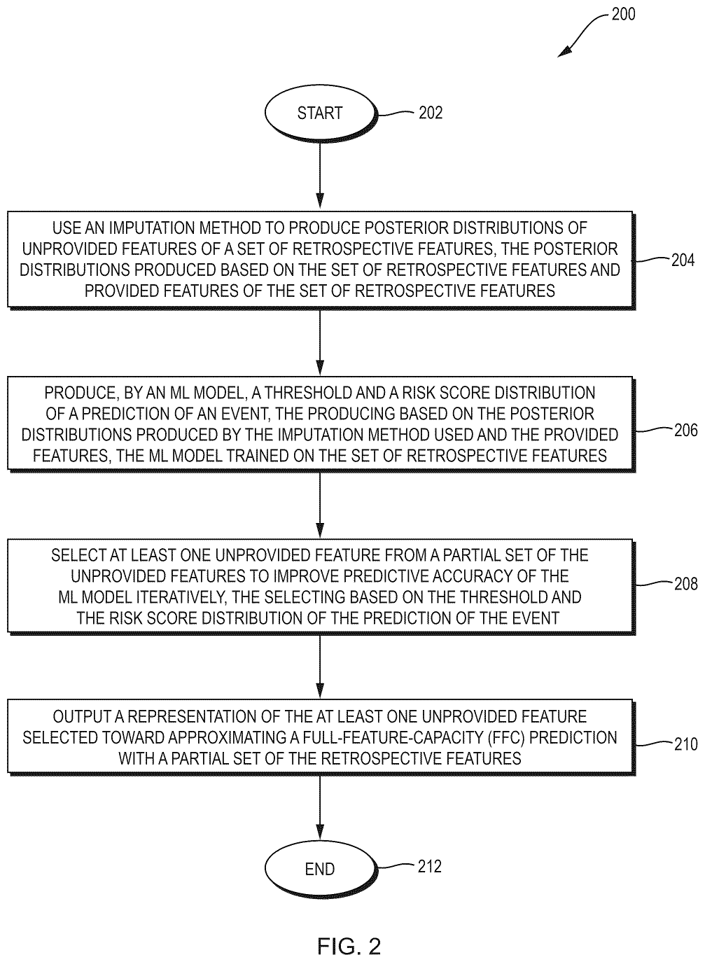

The ML model may be a supervised ML model for non-limiting example. An example embodiment of a computer-implemented method for feature-based ML model prediction that may be implemented by the computer-based system 110 is disclosed below in reference to FIG. 2.

FIG. 2 is a flow diagram of an example embodiment of a computer-implemented method for feature-based machine learning (ML) model prediction (200). The computer-implemented method begins (202) and comprises using an imputation method to produce posterior distributions of unprovided features of a set of retrospective features (204). The posterior distributions are produced based on the set of retrospective features and provided features of the set of retrospective features. The computer-implemented method further comprises producing, by an ML model, a threshold and a risk score distribution of a prediction of an event (206). The producing is based on the posterior distributions produced by the imputation method used and the provided features. The ML model is trained on the set of retrospective features. The computer-implemented method further comprises selecting at least one unprovided feature from a partial set of the unprovided features to improve predictive accuracy of the ML model iteratively (208) The selecting is based on the threshold and the risk score distribution of the prediction of the event. The computer-implemented method further comprises outputting a representation of the at least one unprovided feature selected toward approximating a full-feature-capacity (FFC) prediction with a partial set of the retrospective features (210). The FFC prediction is based on the set of retrospective features in its entirety. The representation output causes the at least one unprovided feature to be provided for a subsequent iteration. The partial set of the retrospective features includes the provided features supplemented by the at least one unprovided feature selected and provided at the subsequent iteration. The computer-implemented method thereafter ends (212) in the example embodiment.

Further technical details are disclosed below.

For non-limiting example, early and timely diagnosis and treatment for diseases is one of the major challenges in medicine. Recent advancements in machine learning (ML) have shown promise in addressing this issue. However, enhancing the performance of these models frequently requires integrating a larger set of clinical features, which can delay prediction and increase healthcare costs. To tackle this limitation, a hypothesis was made that not all patients require the extraction of all clinical variables to make confident decisions. An example embodiment disclosed herein may employ full-feature-capacity (FFC) prediction, referring to that a prediction is made for a patient using only a subset of available features and additional features would not alter the prediction. Then, it can be said that this patient reaches FFC prediction. Disclosed herein are example embodiments of a Feature Sufficiency Analysis (FSA) system, a computational framework designed to determine whether a subset of features is sufficient for an artificial intelligence (AI) model to deliver an FFC prediction. The FSA system may apply the Monte Carlo method to map the effect of missing variables to a risk score distribution, such as the risk score distribution 118 of FIG. 1, disclosed above. The FSA system provides an individualized assessment for the necessity of obtaining unavailable (unprovided) features, thus reducing time and monetary costs associated with feature acquisition. Provided herein are two case studies, postoperative prolonged ventilation prediction for a heart surgery patient cohort and 10-year mortality prediction for an outpatient cohort. It is shown that 86% of the heart surgery cohort and 91% of outpatient cohort require fewer than half of features to reach FFC prediction. A significant time and monetary cost can be reduced while maintaining the FFC prediction. The FSA system also can be used for feature importance ranking and patient grouping, identifying hard-to-predict patient groups where the ML model has less performance drops. Particularly, the performance of the hard-to-predict patient group for 10-year mortality prediction is almost a random guess. The FSA system, which is model-agnostic and tailored to individual patients, offers a novel method to optimize feature utilization in ML models. In summary the FSA system is easy-to-use and cost-saving tool, and useful for an AI application in healthcare or any other field of use.

### INTRODUCTION

Early and timely diagnosis and treatment for diseases is one of the major challenges in medicine. From children acute appendicitis (Cappendijk, V., Hazebrock, W. J. & Hazebrock. The impact of diagnostic delay on the course of acute appendicitis. Arch. Dis. Child. 83, 64-66 (2000)) to sepsis (Husabø, G. et al. Early diagnosis of sepsis in emergency departments, time to treatment, and association with mortality: An observational study. PLOS One 15, c0227652 (2020)), from acute trauma (Vles, W. J., Veen, E. J., Roukema, J. A., Meeuwis, J. D. & Leenen, L. P. H. Consequences of delayed diagnoses in trauma patients: a prospective study: a prospective study. J. Am. Coll. Surg. 197, 596-602 (2003)) to lung cancer (Christensen, E. D., Harvald, T., Jendresen, M., Aggestrup, S. & Petterson, G. The impact of delayed diagnosis of lung cancer on the stage at the time of operation. Eur. J. Cardiothorac. Surg. 12, 880-884 (1997)), studies have shown that delayed diagnosis leads to an increase of complications and/or mortality.

There is an emerging body of work on the use of machine-learning (ML) and other methods to learn models from patient data that make early prediction of disease onset or disease progression, improve accuracy of disease diagnosis, and better inform timely choice of therapy (Domb, B. G. et al. Personalized medicine using predictive analytics: A machine learning-based prognostic model for patients undergoing hip arthroscopy. Am. J. Sports Med. 50, 1900-1908 (2022)), Eloranta, S. & Boman, M. Predictive models for clinical decision making: Deep dives in practical machine learning. J. Intern. Med. 292, 278-295 (2022)), (Fackler, J. C., Rehman, M. & Winslow, R. L. Please welcome the new team member: The algorithm: The algorithm. Pediatric Critical Care Medicine vol. 20 1200-1201 (2019)), (Topol, E. Deep medicine: how artificial intelligence can make healthcare human again. (2019)). Many studies show the impressive extent to which models can help improve disease diagnosis, prediction and treatment (Domb, B. G. et al. Personalized medicine using predictive analytics: A machine learning-based prognostic model for patients undergoing hip arthroscopy. Am. J. Sports Med. 50, 1900-1908 (2022)), (Eloranta, S. & Boman, M. Predictive models for clinical decision making: Deep dives in practical machine learning. J. Intern. Med. 292, 278-295 (2022)), (Topol, E. Deep medicine: how artificial intelligence can make healthcare human again. (2019)), (Wagle, N. et al. aEYE: A deep learning system for video nystagmus detection. Front. Neurol. 13, 963968 (2022)), (Kim, H. B. et al. Computational signatures for post-cardiac arrest trajectory prediction: Importance of early physiological time series. Anaesth Crit Care Pain Med 41, 101015 (2022)), (Liu, R. et al. Prediction of impending septic shock in children with sepsis. Crit. Care Explor. 3, c0442 (2021)), (Krachman, J. A. et al. Predicting flow rate escalation for pediatric patients on high flow nasal cannula using machine learning. Front. Pediatr. 9, 734753 (2021)), (Bosc, S. N. et al. Early prediction of multiple organ dysfunction in the pediatric intensive care unit. Front. Pediatr. 9, 711104 (2021)), (Liu, R., Greenstein, J. L., Fackler, J. C., Bembea, M. M. & Winslow, R. L. Spectral clustering of risk score trajectories stratifies sepsis patients by clinical outcome and interventions received. Elife 9, e58142 (2020)), (Liu, R. et al. Data-driven discovery of a novel sepsis pre-shock state predicts impending septic shock in the ICU. Sci. Rep. 9, 6145 (2019)), (Seol, H. Y. et al. Artificial intelligence-assisted clinical decision support for childhood asthma management: A randomized clinical trial. PLOS One 16, e0255261 (2021)), (Yao, X. et al. ECG AI-Guided Screening for Low Ejection Fraction (EAGLE): Rationale and design of a pragmatic cluster randomized trial. Am. Heart J. 219, 31-36 (2020)), (Richens, J. G., Lee, C. M. & Johri, S. Improving the accuracy of medical diagnosis with causal machine learning. Nat. Commun. 11, 3923 (2020)), (Shimabukuro, D. W., Barton, C. W., Feldman, M. D., Mataraso, S. J. & Das, R. Effect of a machine learning-based severe sepsis prediction algorithm on patient survival and hospital length of stay: a randomised clinical trial. BMJ Open Respir. Res. 4, e000234 (2017)), (Esteva, A. et al. Dermatologist-level classification of skin cancer with deep neural networks. Nature 542, 115-118 (2017)), (Gulshan, V. et al. Development and validation of a deep learning algorithm for detection of diabetic retinopathy in retinal fundus photographs. JAMA 316, 2402-2410 (2016)), (Annapragada, A. V. et al. SWIFT: A deep learning approach to prediction of hypoxemic events in critically-Ill patients using SpO2 waveform prediction. PLOS Comput. Biol. 17, e1009712 (2021)), including using images to classify cancer (Esteva, A. et al. Dermatologist-level classification of skin cancer with deep neural networks. Nature 542, 115-118 (2017)), using longitudinal ICU data to predict septic shock (Liu, R. et al. Data-driven discovery of a novel sepsis pre-shock state predicts impending septic shock in the ICU. Sci. Rep. 9, 6145 (2019)), using waveform data to predict neurological outcome (Kim, H. B. et al. Computational signatures for post-cardiac arrest trajectory prediction: Importance of early physiological time series. Anaesth Crit Care Pain Med 41, 101015 (2022)), using clinical code to predict the pancreatic cancer occurrence 3 month ahead, using lab result data to diagnose ovarian cancer (Cai, G. et al. Artificial intelligence-based models enabling accurate diagnosis of ovarian cancer using laboratory tests in China: a multicentre, retrospective cohort study. Lancet Digit. Health 6, e176-c186 (2024)) and use multimodal data to predict severity of COVID-19 patients (Lassau, N. et al. Integrating deep learning CT-scan model, biological and clinical variables to predict severity of COVID-19 patients. Nat. Commun. 12, 634 (2021)). Some studies examine performance in real-time clinical applications (Venema, E. et al. Large-scale validation of the prediction model risk of bias assessment Tool (PROBAST) using a short form: high risk of bias models show poorer discrimination. J. Clin. Epidemiol. 138, 32-39 (2021)), (Wessler, B. S. et al. External validations of cardiovascular clinical prediction models: A large-scale review of the literature. Circ. Cardiovasc. Qual. Outcomes 14, e007858 (2021)).

However, enhancing the performance of these ML models often involves integrating a more extensive set of features. For example, models that utilize multimodal data have been shown to outperform those using a single modality (Kim, H. B. et al. Computational signatures for post-cardiac arrest trajectory prediction: Importance of early physiological time series. Anaesth Crit Care Pain Med 41, 101015 (2022)). Soenksen et al. systematically demonstrated that multimodal models have yielded consistent improvement of performance across various healthcare applications, from lung lesion prediction to 48-hour mortality (Soenksen, L. R. et al. Integrated multimodal artificial intelligence framework for healthcare applications. NPJ Digit. Med. 5, 149 (2022)). Obtaining more clinical data will then, again, lead to a delayed prediction. For example, the turnaround time (TAT) for the complete blood count (CBC) test is ˜30 mins with the highest urgency (Lou, A. H. et al. Multiple pre- and post-analytical lean approaches to the improvement of the laboratory turnaround time in a large core laboratory. Clin. Biochem. 50, 864-869 (2017)). TAT for brain MRI ranges from 40-224 mins (Hayatghaibi, S. E. et al. Turnaround time and efficiency of pediatric outpatient brain magnetic resonance imaging: a multi-institutional cross-sectional study. Pediatr. Radiol. 53, 1144-1152 (2023)). Additionally, an extensive data extraction will lead to an increase in healthcare cost. Rising costs of healthcare services remains one of the major challenges in the healthcare industry (Folland, S., Goodman, A. C., Stano, M. & Danagoulian, S. The Economics of Health and Health Care. (Routledge, London, England, 2024)).

Given those limitations, a hypothesis was made that not every patient requires to extract all clinical variables, namely features, to make confident decisions. That is to say, the number of features needed to achieve an accurate diagnosis may vary: some patients may only need a small subset of features because the signal of those features are sufficiently strong such that confident predictions can be made. When considering a ML model, a full feature list may be essential for making a confident prediction for some patients, whereas a handful of features are enough for others. A hypothesis was made that not every patient requires the entire feature list to enable the model to predict with its full capacity. Herein, a prediction with a full set of features may be defined as full-feature-capacity (FFC) prediction. A confident prediction for a patient with a subset of features represents that, regardless of what the other features values are, the prediction will remain the same. Such prediction can be said to reach the FFC prediction, or that this is a FFC-predictable patient.

Clinical features that are not available due to either lab TAT, monetary cost or other reasons, can be treated as missing values. A vast amount of work on missing value imputation has been done (Hasan, M. K. et al. Missing value imputation affects the performance of machine learning: A review and analysis of the literature (2010-2021). Inform. Med. Unlocked 27, 100799 (2021)) from mean to ML-based imputation methods. Imputation techniques enable ML models to make inferences. However, the model performance will almost certainly drop on samples with missing values and there is no principled method to estimate the decrease of performance.

To address those issues, an example embodiment of a computational framework, referred to as a Feature Sufficiency Analysis (FSA) system, may be designed to ascertain whether a subset of features is sufficient for the AI model to deliver a prediction with full feature capacity (FFC). FFC may be defined as a prediction made with a complete set of features. If a patient has only a portion of features available, and the prediction remains the same regardless of the possible values for the missing variables, an example embodiment may consider that this patient with the subset of features reaches the FFC. This framework is developed based on Bayesian approach alongside uncertainty analysis to determine the impact of missing features on the model's predictive accuracy. It is shown with two case studies that this framework is able to reduce the time and monetary cost for feature acquisition. Rather than following the imputation-prediction ML paradigm, this framework performs the Bayesian-based multiple imputation and then evaluates the necessity to obtain unavailable (unprovided) features for each patient. This tool is model-agnostic, ensuring compatibility across various AI models, and it generates tailored inference to each patient. Following FFC prediction, the FSA system provides intuitive tools to perform two fundamental tasks in the machine learning field. 1. Feature importance ranking. The FSA-based feature ranking was validated with widely-used feature ranking methods on the case studies. 2. Patient grouping, the FSA system can identify the hard-to-predict patient group, where the ML performance dramatically drops in this group. In summary, the FSA system is a principled, easy-to-use and important tool to make healthcare risk models, or any other ML model, cost-effective while preserving the model performance.

### Results.

### Prediction Tasks and Baseline AI Model.

Disclosed herein are two clinical prediction tasks that were performed. The first task is to develop an AI model to predict the postoperative prolonged ventilation for heart surgery patients. The definition of prolonged ventilation is that the postoperative prolonged ventilation is greater than >24 hrs. The STS Adult Cardiac Surgery Database (ACSD) (Bowdish, M. E. et al. STS Adult Cardiac Surgery Database: 2021 update on outcomes, quality, and research. Ann. Thorac. Surg. 111, 1770-1780 (2021)) was queried to develop a dataset including cardiac surgery cases from a single center over a 10-year period from Jan. 1, 2012 to Dec. 31, 2021, that included 9,238 patients. 12 features disclosed in Table 1, below, are extracted to make the baseline model.

TABLE 1
12 features for the baseline ML model.

| Acronyms | Description [categorical values] |
|---|---|
| iabpwhen | When the IABP was inserted |
| | [No, Yes, preoperative, Yes intraoperative] |
| creatlst | Indicate the creatinine level closest to the date and time prior surgery |
| age | |
| hdef | Ejection fraction |
| bmi | Body mass index |
| status | Clinical status of the patient prior to entering the operating room. |
| | [Elective, Urgent, Emergent, Emergent Salvage] |
| hct | Indicate the pre-operative Hematocrit level at the date and time closest to surgery |
| lwsthct | The lowest measured hematocrit recorded in the operating room. |
| carshock | If the patient developed cardiogenic shock. |
| wbc | the pre-operative White Blood Cell (WBC) count closest to the date and time |
| | prior to surgery but prior to anesthetic management |
| platelets | Platelet count closest to the date and time prior to surgery but prior to anesthetic |
| | management |
| vdinsufm | Whether there is evidence of Mitral valve insufficiency/regurgitation. |
| | [None, Trivial/Trace, Mild. Moderate, Severe, Not documented] |

The data are stratified sampling into training (70%), validation (10%) and test set (20%). Random forest is used to generate a baseline AI model, achieving 0.88 AUC, 0.67 sensitivity, 0.94 specificity, 0.94 positive predictive value (PPV) and the threshold is 0.285. If the predicted risk score is greater than the threshold, the model makes a positive prediction.

The second task is to predict the 10-year mortality for an outpatient cohort from the long-running National Health and Nutrition Examination Survey (NHANES) with 13,442 outpatients and 35 features across the United States (Miller, H. W. Plan and operation of the health and nutrition examination survey. United states—1971-1973. Vital Health Stat. 1 1-46 (1973)). The data is publicly available and organized from Erion et al. (Erion, G. et al. A cost-aware framework for the development of AI models for healthcare applications. Nat Biomed Eng 6, 1384-1398 (2022)).

Random forest was also applied to develop a baseline AI model, achieving 0.86 AUC, 0.79 sensitivity, 0.79 specificity, 0.92 PPV and the threshold is 0.696. A detailed description of the model and features are described further below in the Methods section.

Two tasks are selected for 3 reasons. 1. Two tasks represent two distinct types of clinical prediction tasks, an acute event prediction happening in highly monitored ICU units and a long-term 10-year mortality prediction. 2. Two tasks both have a measurement of feature costs. 3. Two tasks represents two distinct types of prediction with respect to the class imbalance: minority outcome (˜10% for prolonged ventilation) and the majority outcome (˜75% for mortality prediction).

### Feature Sufficiency Analysis (FSA) System.

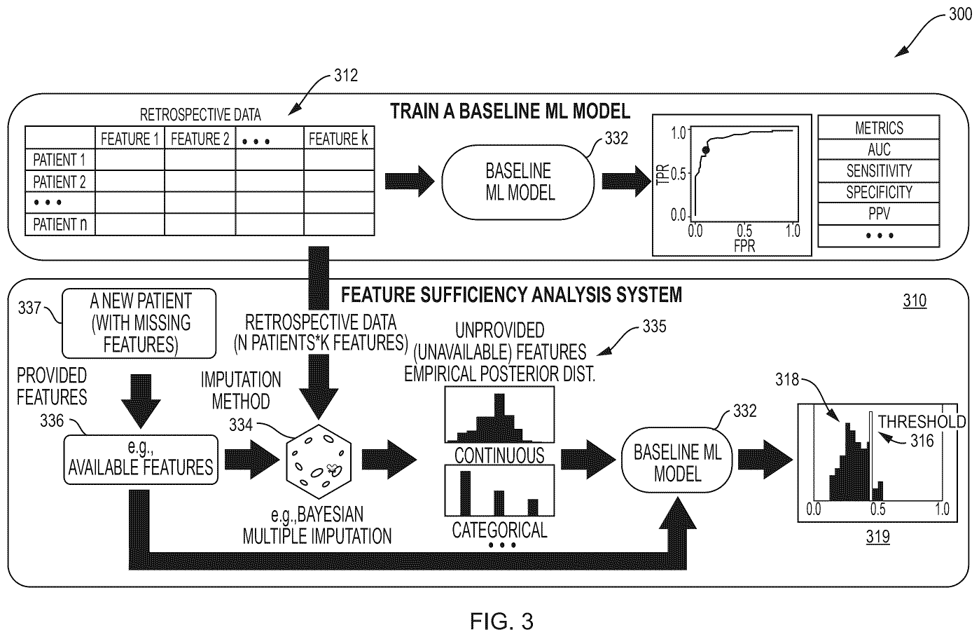

FIG. 3 is a block diagram 300 of an example embodiment of a feature sufficiency analysis (FSA) system 310 that may be employed in the computer-based system 110 of FIG. 1, disclosed above. Continuing with reference to FIG. 3, the FSA system 310 is designed to evaluate whether a full-feature-capacity (FFC) prediction is feasible using a subset of patient features. Although two case studies were applied and used with the random forest baseline model for demonstration, the FSA system 310 can be applied to any predictive task and ML methods. In FIG. 3, first, a baseline ML model is developed based on the retrospective data of K features and N patients, that is, a set of retrospective features 312. The FSA system 310 is trying to ask the following question, for a new patient with only k′ K features available (provided), can the ML model 332 make an FFC prediction despite the uncertainty from missing features? The FSA system 310 may quantify the uncertainty of unavailable features by applying an imputation method 334, such as MICE: Multiple Imputation by Chained Equations (Royston, P. & White, I. R. Multiple Imputation by Chained Equations (MICE): Implementation in Stata. J. Stat. Softw. 45, 1-20 (2011)) for non-limiting example. MICE is a widely used Bayesian-based multiple imputation method. MICE estimates the posterior distribution of unavailable features using Monte Carlo method based on the historical data, namely the data of the set of retrospective features 312, and available (provided) features 336, for example, for a new patient 337 with missing features. For the new patient 337, the baseline model 332 input includes a set of available feature values, that is, the provided features 336, and inferred posterior distributions 335 for the unprovided (unavailable) features. Such ML model input propagates through the baseline ML model 332 to yield a distribution of risk score, that is, a risk score distribution 318. The risk score distribution 318 is assessed against the predetermined threshold 316 on the risk score 319. If the entirety of the distribution surpasses or is below this threshold 316, it indicates that the current feature set, that is, the provided features 336, suffices for the ML model 332 to make a FFC prediction despite the uncertainty of unobserved features. In practice, the risk score distribution 318 may be approximated with Monte Carlo (MC) methods, and 100 realizations were generated to generate an empirical distribution. Thus, if all 100 realizations greater or less than the threshold, FFC prediction is achieved. In contrast, if the risk score distribution 318 intersects with the threshold 316, this uncertainty highlights the need for and importance of additional features to obtain the accurate prediction. A detailed description can be found in the Methods section disclosed further below.

Most Patients Require Fewer than Half of Features to Reach Full-Feature-Capacity Prediction.

Continuing with FIG. 3, the FSA system 310 can provide feature importance ranking by performing the ablation study. Ablation study is to remove a feature from the data to see how much drops of the model performance (Hameed, I. et al. BASED-XAI: Breaking ablation studies down for explainable artificial intelligence. arXiv [cs.LG] (2022)). An example embodiment may treat the removed feature as missing such that the FSA system 310 estimates the uncertainty (posterior distribution) of the removed feature and the corresponding risk score distribution 318. The number of patients whose risk score distribution intersects the threshold was counted. A greater number of patients intersecting the threshold 316 indicates that the removed feature is essential to make prediction such that the removed feature is more important. Each feature was removed from the test set. The FSA system 310 estimated the uncertainty of the removed feature.

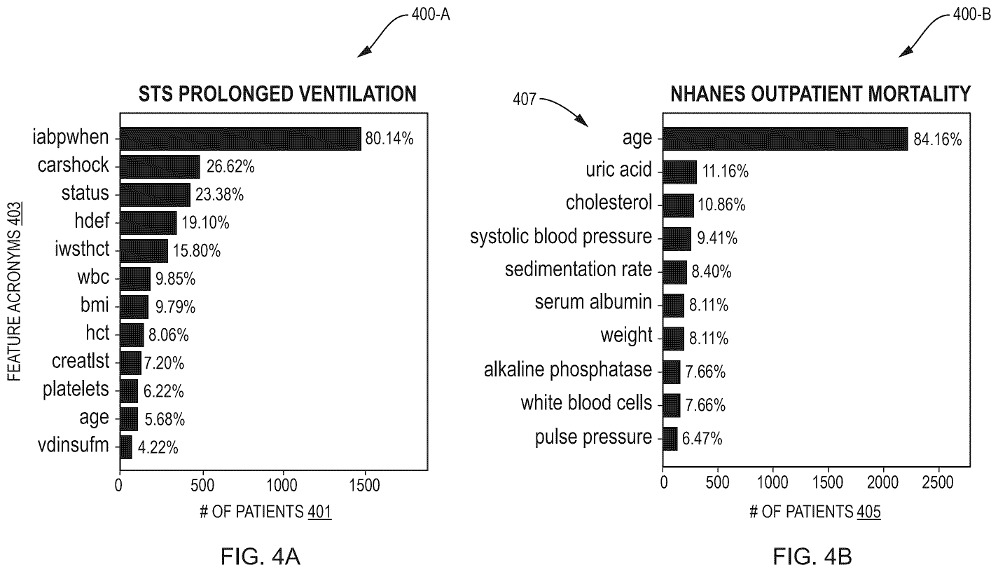

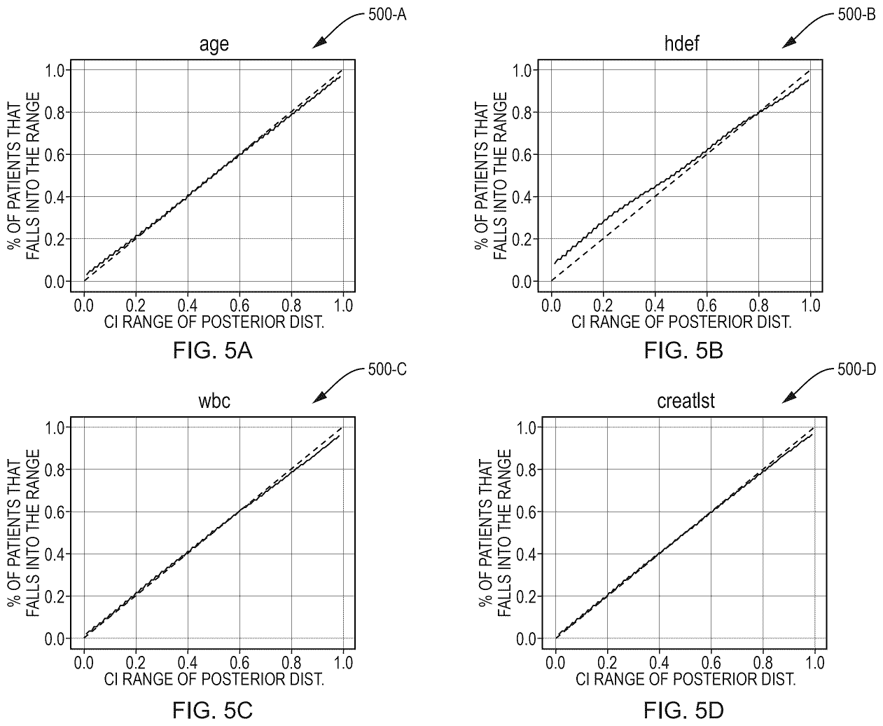

The percentage and count of patients that are FFC-predictable are presented as a measure of feature importance in FIG. 4A and FIG. 4B.

### Evaluating the Necessity of Features for FFC Prediction.

FIG. 4A is a graph 400-A of an example embodiment of Society of Thoracic Surgeons (STS) prolonged ventilation prediction per number of patients 401 and features, noted by feature acronyms 403.

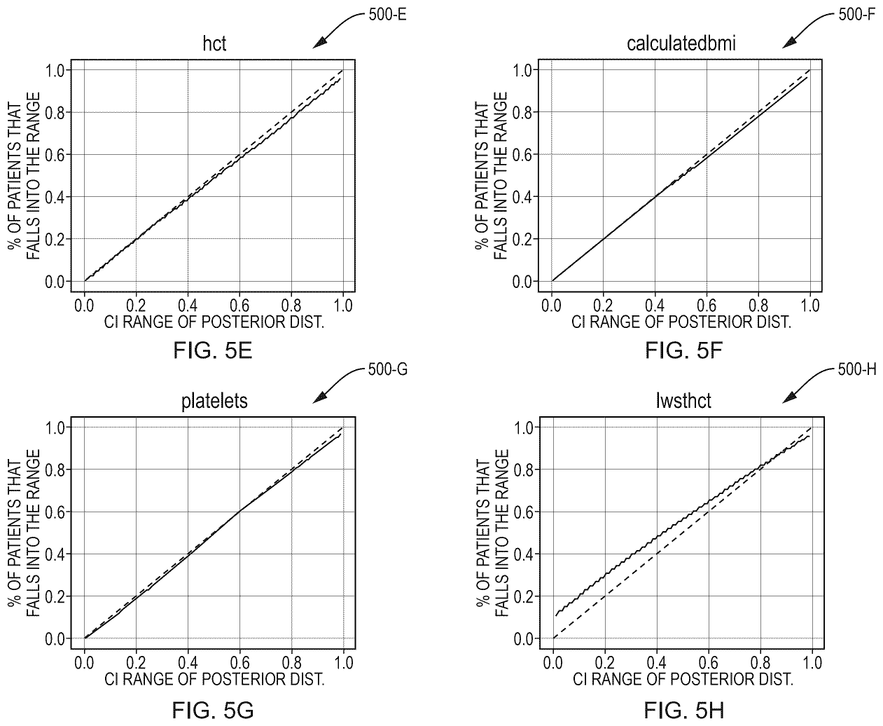

FIG. 4B is a graph 400-B of an example embodiment for 10-year mortality prediction per number of patients 405 and features 407. The graphs 400-A and 400-B evaluate the necessity of features for FFC prediction. The graph 400-A is for the prolonged ventilation prediction and the graph 400-B is for the 10-year mortality prediction. Feature importance is shown for features (top 10 features for mortality prediction) by assessing the number of patients (n) and percentage (%) for whom FFC prediction cannot be achieved when specific features are removed. The estimation of the FSA-generated uncertainty posterior distribution was validated, as shown in FIGS. 5A-T.

FIGS. 5A-T are graphs (500-A, . . . , 500-T) of results that illustrate the validation of the estimated posterior distribution on STS heart surgery patient cohort. The posterior distribution generated from the ablation study for the feature ranking are examined. For continuous variables (age, hdef, wbc, creatlst, hct, bmi, platelets and lwsthct), the posterior distribution is validated by comparing the credible intervals (x-axis) and the percentage of patients falling into the credible intervals. For categorical variables (iabpwhen, status, carshock and vdinsufm), calibration is plotted for each unique value for categorical variables, where the x-axis is the predicted probability of the value, and the y-axis is the % of patients having this value.

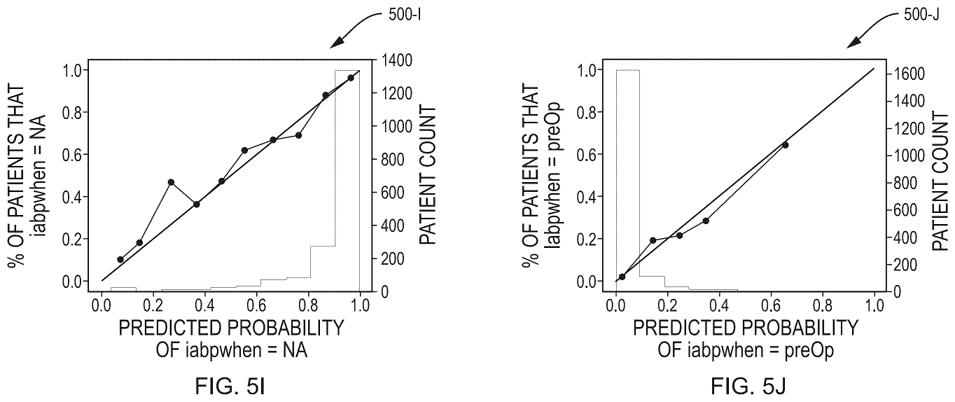

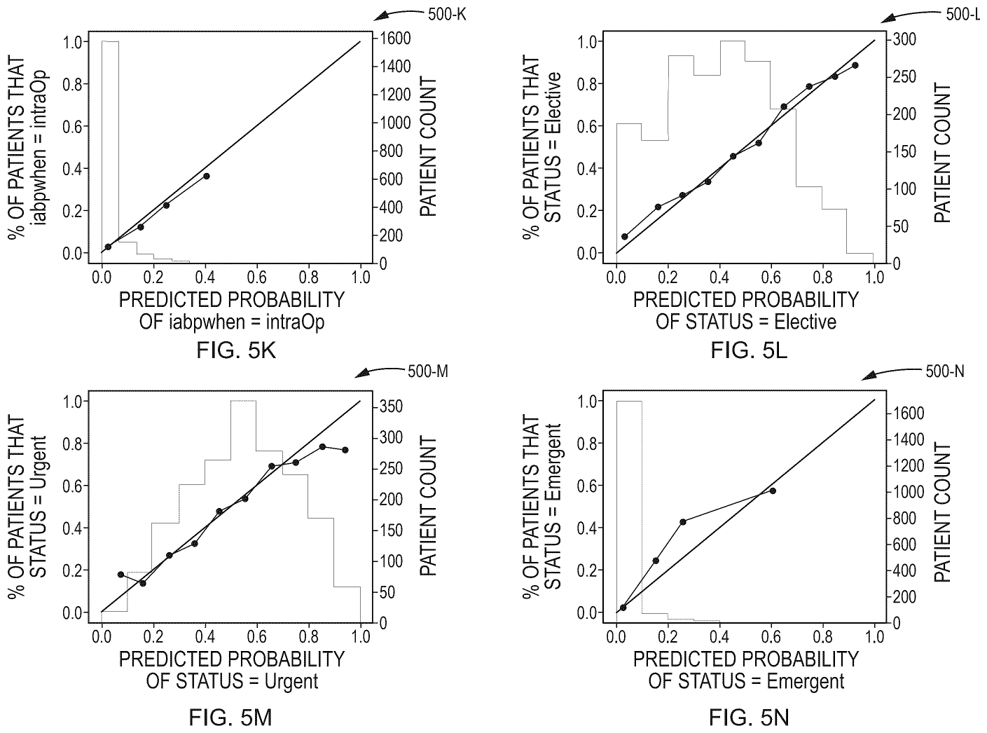

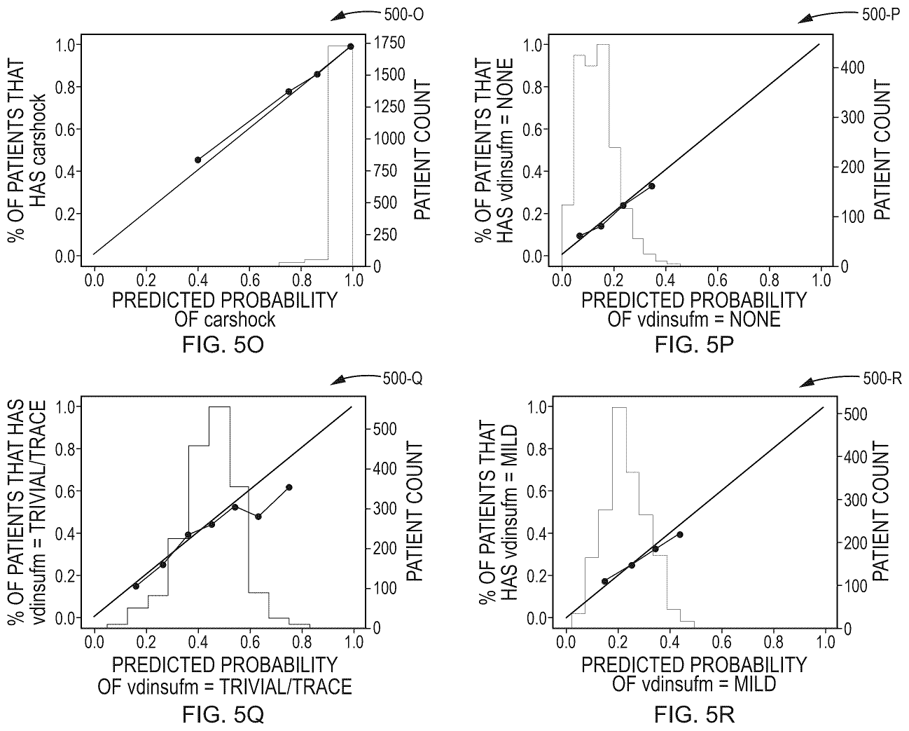

An example embodiment of an FSA-based feature ranking method disclosed herein was compared and consistency shown with the widely-used feature ranking methods, as disclosed in FIGS. 6A-C.

FIG. 6A is a graph 600 of an example embodiment of a comparison between FSA-based feature ranking with 4 widely-used ranking methods. In FIG. 6A, the cumulative feature sufficiency analysis for the STS prolonged ventilation prediction based on 5 different feature order including the order of FSA-based feature ranking 611 and 4 widely used ranking methods: mean decrease of impurity (MDI) 613, permutation ranking method 615, Shapley value 617, and logistic regression 619. The feature order for the cumulative feature sufficiency analysis is based on the feature ranking, the x-axis is the first n features available 621 based on the feature ranking order. The y-axis is the % of FFC-predictable patients 623.

FIG. 6B is a table 600-B of an example embodiment of a pairwise spearman correlation between the different ranking methods of FIG. 6A.

FIG. 6C is a table 600-C that summarizes the five different feature ranking methods of FIGS. 6A and 6B.

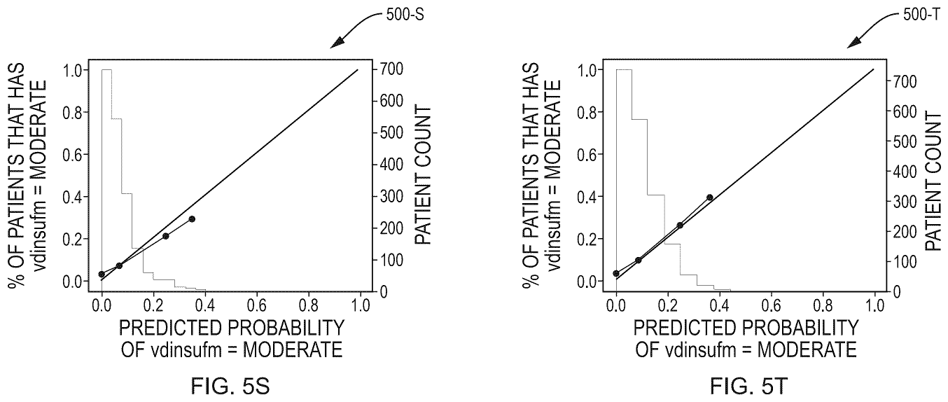

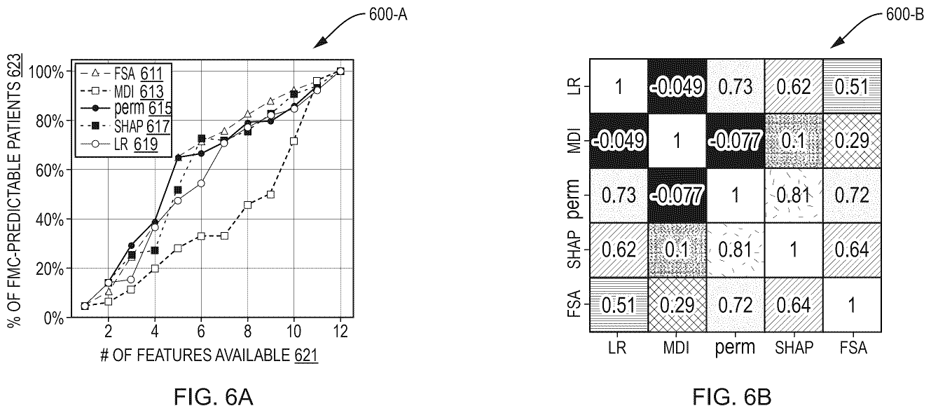

With reference back to FIG. 3 and following with the feature ranking from most to least important, features were iteratively included into the patient data. Every time, one feature is added, and the FSA system 310 will decide if a FFC-prediction can be made. This analysis is defined herein as cumulative feature sufficiency analysis, described in the Methods section further below. Once an FFC-prediction is achieved for a patient for the first time in the iterative feature inclusion process, the number of available features is the minimum number of necessary features for this patient to reach FFC prediction. FIGS. 7A and 7B show the distribution of the minimum number of necessary features.

FIGS. 7A and 7B are graphs (700-A, 700-B) of a distribution of a minimum number of features needed to make an FFC prediction. In FIG. 7A, the graph 700-A is for prolonged ventilation prediction. In FIG. 7B, the graph 700-B is for a 10-year mortality prediction.

FIGS. 7C-H are boxplots (700-C, . . . , 700-H) for 6 patient examples and demonstrate the evolution of risk score distribution with an increasing number of features available. The boxplots 700-C, 700-E, and 700-G are for prolonged ventilation prediction. The boxplots 700-D, 700-F, and 700-H are for the 10-year mortality prediction. The x-axis represents the number of available (provided) features. The horizontal red lines (723-C, 723-D, 723-E, 723-F, 723-G, and 723-H) shows the threshold of the ML model and the red stars (725-C, 725-D, 725-E, 725-F, 725-G, and 725-H) show the FFC-prediction wherein all features are available (provided). The inclusion order of features for FIGS. 7C-H follows the feature ranking in FIG. 4A and FIG. 4B.

With reference to FIGS. 7C-H, the graphs 700-C through 700-H show the risk score distribution of 3 distinct patterns of patient samples: 1) FIG. 7C and FIG. 7D, negative prediction that FFC prediction can be achieved with very few features, 2) FIGS. 7E and 7F, positive prediction that FFC prediction can be achieved with very few features, and 3) FIGS. 7G and 7H, prediction that closes to the threshold and almost all features are necessary to achieve the FFC prediction. These results provide a useful message that a majority of patients don't need all variables to achieve the FFC prediction.

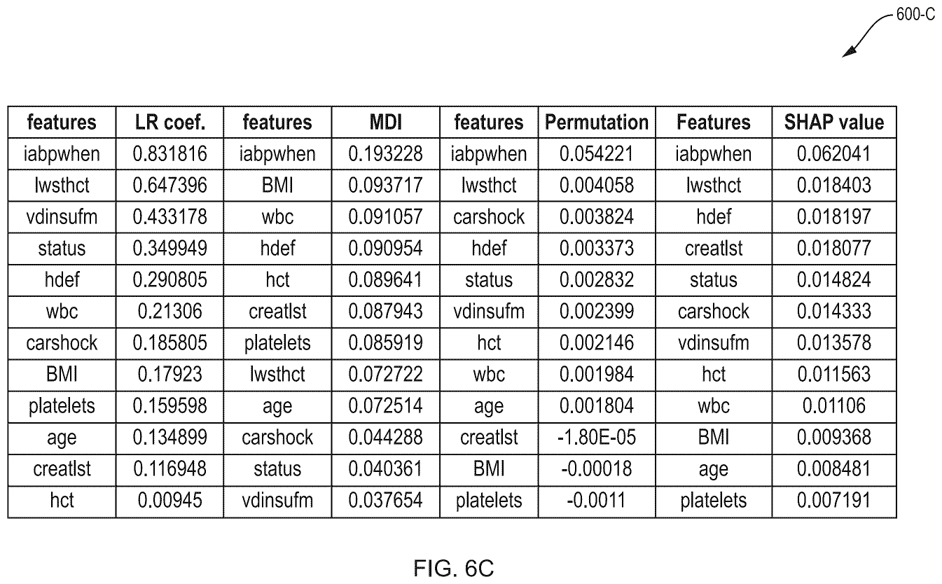

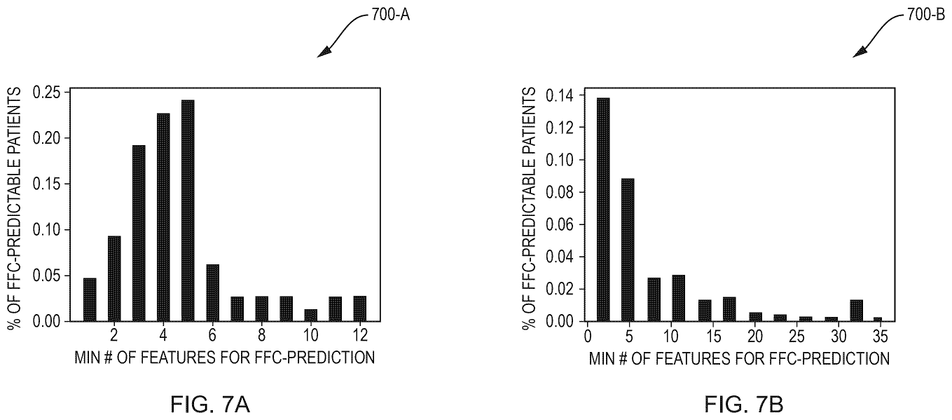

FIG. 8A is a graph 800-A of an example embodiment of cumulative feature sufficiency analysis for prolonged ventilation.

FIG. 8B is a graph 800-B of an example embodiment of cumulative feature sufficiency analysis for mortality prediction. FIGS. 8A and 8B show the percentage of FFC-predictable patients with top n important features from FSA-ranking. Notably, ˜86% patients for prolonged ventilation task and ˜91% patients for 10-year mortality prediction require less than or equal to half of features to achieve FFC prediction. FIGS. 8A and 8B show that following the FSA-based feature ranking, the % of FCC-predictable patients with the top n features can be identified based on the ranking from the most to least important.

### Feature Cost and the FFC Prediction.

The time and monetary cost of features annotated by 2 clinical practitioners for STS prolonged ventilation prediction is shown in Table 2, below.

TABLE 2
Time and monetary cost for 12 STS features

| | | | Monetary |
|---|---|---|---|
| Testing/Action | Variables | Time cost | cost |
| CMP | creatlst | 15 mins-1 hr | $277 |
| CBC | lwsthct, wbc, hct, | 15 mins-1 hr | $119 |
| | platelets | | |
| Echo ultrasound | vdinsufm, hdef | 50 hrs-4 wks | $573 |
| Demographics | bmi, age | 15 s | $0 |
| History & Physical | carshock | 15 s | $0 |
| Procedure | iabpwhen | 15 s | $0 |
| Chart review | status | 15 s | $0 |

Features are grouped by the clinical test/actions. The cost analysis was performed, similar to cumulative feature sufficiency analysis, except that 1. An example embodiment iteratively included clinical test/action potentially composed of multiple features. Ex. Echo ultrasound is one clinical test generating both hdef (ejection fraction) and vdinsufm (Mitral valve insufficiency/regurgitation). 2. Rather than showing the first n features, cumulative time/monetary cost of available clinical test/action are shown in the plot. A detailed description of cost analysis can be found in the Methods section disclosed further below. The cost analysis is based on the cost order of clinical test/action from least to the most.

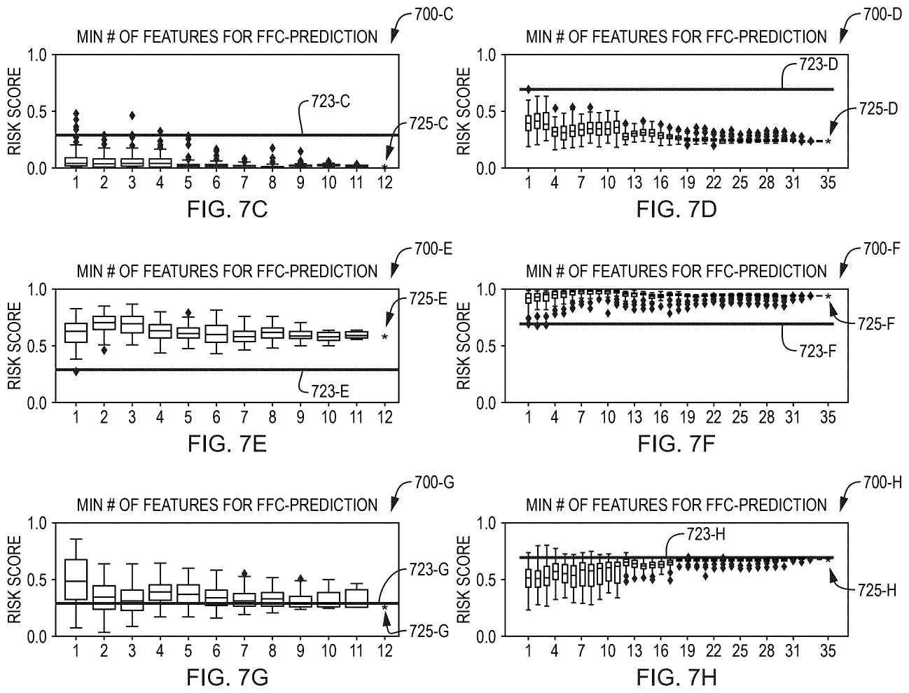

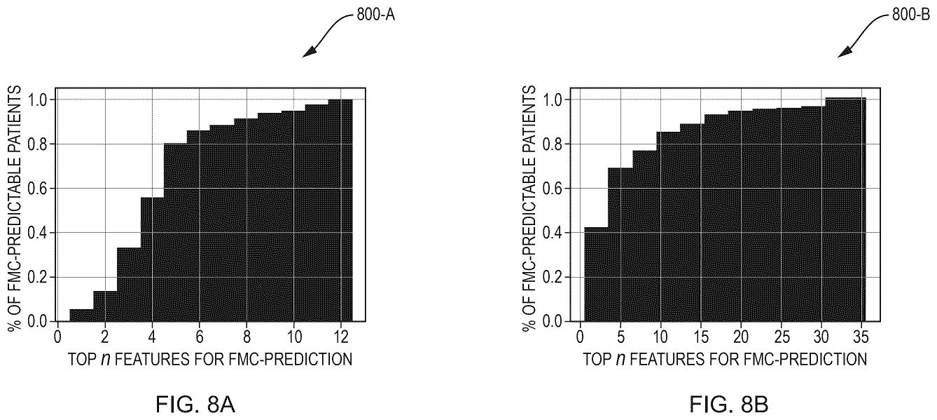

FIG. 9A is graph 900-A of an example embodiment of a percentage of FFC-predictable patients given time.

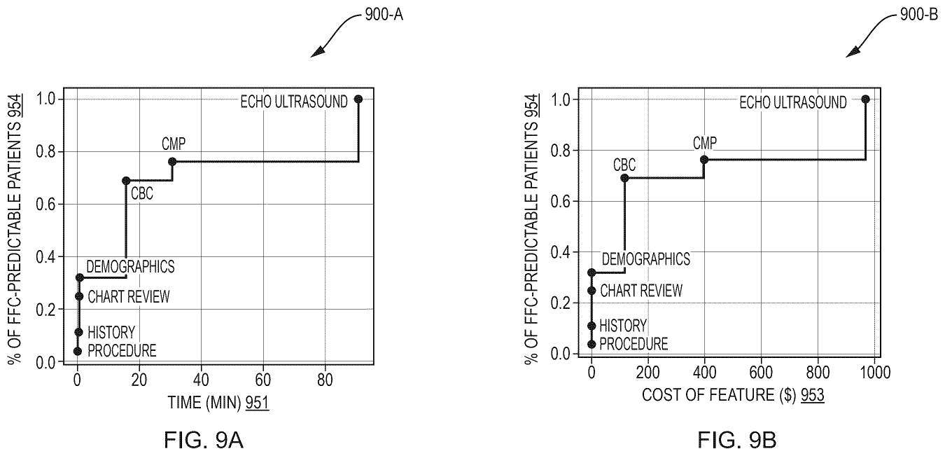

FIG. 9B is graph 900-B of an example embodiment of a percentage of FFC-predictable patients given monetary cost. As shown, ˜75% patients can reach FFC prediction without the echo ultrasound, the most expensive clinical test for time and money. Notably, echo ultrasound accounts for more than half of the time and monetary cost. A similar conclusion also exists in the monetary cost analysis on the NHANES 10-year mortality. FIGS. 9A and 9B shown time (FIG. 9A) and monetary (FIG. 9B) cost analysis on the prolonged ventilation prediction. By iteratively adding clinical test/actions to the ML model, the x-axis shows the cumulative time 951 and monetary cost 953, and the y-axis shows the percentage of patients 954 that can reach FFC prediction given the cost. The clinical test/actions are iteratively added based on the cost order from the least to the most. Each clinical test/action is composed of one or more features. prediction shown in FIG. 10, disclosed below. These indicate that a significant resource can be saved by an example embodiment of an FSA system disclosed herein.

FIG. 10 is a graph 1000 of an example embodiment of a cost analysis for the 10-year mortality. The graph 1000 shows a % of FFC-predictable patients 1041 with the increase of monetary cost 1043 of features for the NHANES cohort.

### FSA System Outperforms the Sub-Model Method on Cohort-Level Performance.

One classical strategy for making predictions with missing variables is to develop a collection of sub-models, where each sub-model is trained with a unique subset of features (Erion, G. et al. A cost-aware framework for the development of AI models for healthcare applications. Nat Biomed Eng 6, 1384-1398 (2022)). Then, for a patient with a subset of variables available, a suitable sub-model will be applied. In contrast, an example embodiment of the FSA system uses one ML model trained with all features, that is, the set of retrospective features in its entirety, and provides a risk score distribution driven by the missing variables. Here, the average of the risk score distribution can be taken and used as the score for prediction.

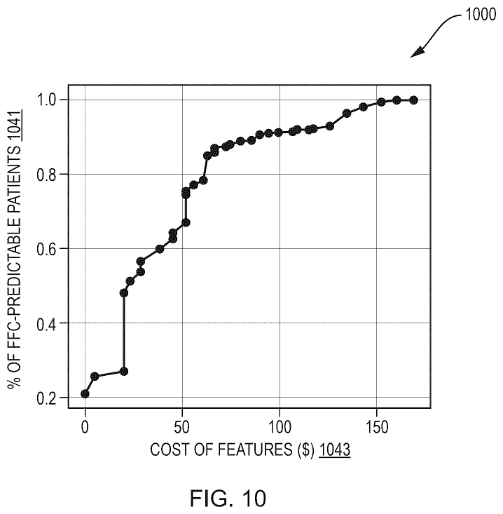

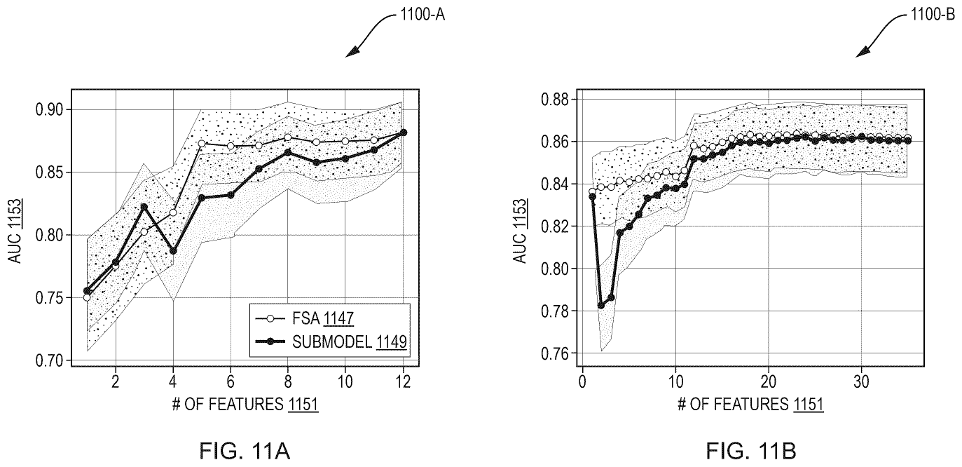

FIG. 11A is a graph 1100-A of ML model performance for subsets of features for prolonged ventilation.

FIG. 11B is a graph 1100-B of ML model performance for subsets of features for mortality prediction. FIGS. 11A and 11B show the comparison between an example embodiment of FSA method (blue) 1147 and sub-model method (red) 1149 with the increase of number of available features 1151 for both prolonged ventilation prediction (FIG. 11A) and mortality prediction (FIG. 11B). Features were added iteratively based on the feature ranking in FIG. 4A and FIG. 4B starting from most to least important. It shows that, for most cases, an example embodiment of an FSA method outperforms the sub-model method. Additionally, the performance of an example embodiment of an FSA method monotonically elevates with the increase of available features, whereas the sub-model method oscillates significantly, particularly when very little features is available. In FIGS. 11A and 11B, the AUC (y-axis with 95% confidence interval) representing the ML performance changes with the increase of the number of features 1151. An example embodiment of the FSA method (blue curve) 1147 uses the average of risk score distribution to represent the prediction with the performance. Alternatively, sub-models (red curve) 1149 are trained for each subset of features and to evaluate the prediction performance.

### Identifying the Hard-to-Predict Patient Group

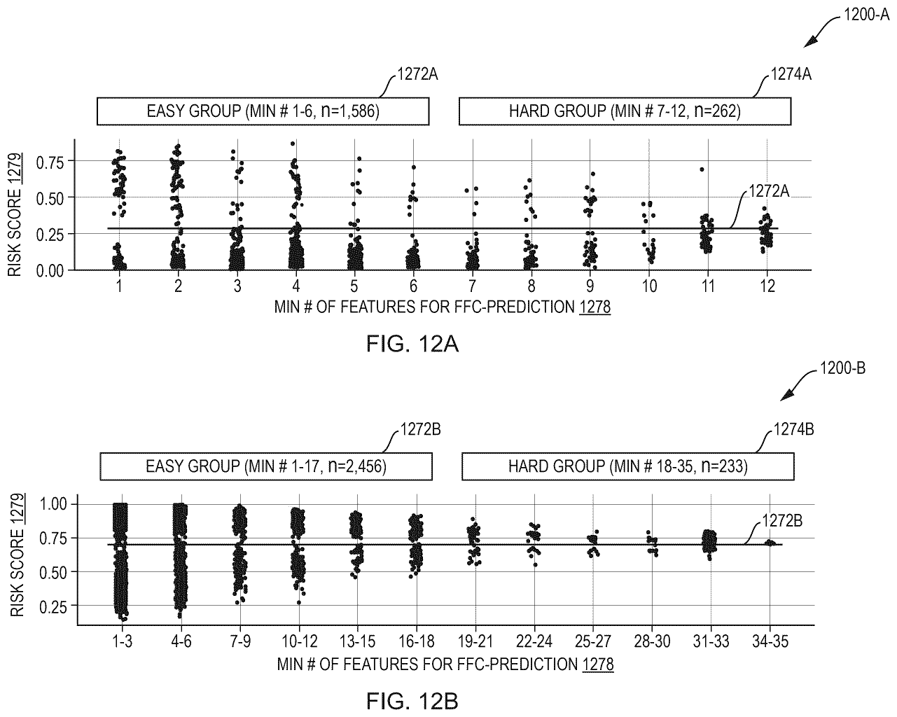

FIGS. 12A-D are graphs (1200-A, 1200-B, 1200-C, and 1200D) of example embodiments of grouping patients into easy and hard groups. Patient phenotyping and clustering is an essential topic in the EHR field (Loftus, T. J. et al. Phenotype clustering in health care: A narrative review for clinicians. Front. Artif. Intell. 5, 842306 (2022)). An example embodiment of an FSA system disclosed herein can identify two patient groups in the dataset based on the minimum number (min #) of necessary features to reach FFC-prediction. The min #features are identified in FIG. 7A and FIG. 7B. FIGS. 12A (for prolonged ventilation prediction) and 12B (for mortality prediction) show the risk scores for patients categorized by min #features. Patients are separated into two groups: easy group (1272a, 1272b): min #necessary features less or equal to total number of features, and hard group (1274a, 1274b): min #necessary features greater than total number of features. Easy group occupies the majority of the cohort (86% for STS heart surgery cohort and 91% for NHANES cohort), and the risk scores reside mostly away from the threshold for binary prediction. FIGS. 12C and 12D show that the easy group achieves significantly higher AUC than the hard group. Particularly, the AUC of the hard group of NHANES cohort is 0.46 (0.38-0.54), indicating that the ML classifier is essentially a random guess for the hard group.

With reference to the graphs 1200-A of FIG. 12A and 1200-B of FIG. 12B, for each patient, the minimum number (#) of necessary features 1278 for FFC prediction was identified. The dot plot of the risk score 1279 categorized by the minimum number of necessary features for FFC prediction is performed for prolonged ventilation prediction on STS heart surgery cohort (FIG. 12A) and 10-year mortality prediction on NHANES cohort (FIG. 12B). The patients were grouped into easy group, defined as #of necessary features < half of total #of features and hard group, defined as #of necessary features > half of total #of features. The horizontal red line (1272a, 1272b) is the threshold for binary prediction. The receiver operating curve with 95% CI is shown in two groups, for prolonged ventilation prediction in the graph 1200-C of FIG. 12C and mortality prediction in the graph 1200-D of FIG. 12D. Tables 3 and 4 below show further that the easy group achieves significantly better performance in sensitivity, specificity, positive predictive value (PPV), negative predictive value (NPV) and accuracy. Thus, the hard group is harder for prediction, thus naming as hard-to-predict group.

TABLE 3
Performance metrics for easy group, hard group and all
patients for prolonged ventilation. mean (95% CI)

| | Easy | Hard | All |
|---|---|---|---|
| AUC | 0.89 (0.85, 0.92) | 0.79 (0.71, 0.86) | 0.88 (0.85, 0.90) |
| Sens | 0.69 (0.61, 0.77) | 0.60 (0.44, 0.74) | 0.67 (0.60, 0.73) |
| Spec | 0.96 (0.95, 0.97) | 0.81 (0.76, 0.86) | 0.94 (0.93, 0.95) |
| PPV | 0.65 (0.58, 0.72) | 0.38 (0.26, 0.50) | 0.57 (0.50, 0.64) |
| NPV | 0.97 (0.96, 0.98) | 0.91 (0.87, 0.95) | 0.96 (0.95, 0.97) |
| Acc | 0.94 (0.92, 0.95) | 0.78 (0.73, 0.82) | 0.91 (0.90, 0.93) |

TABLE 4
Performance metrics for easy group, hard group and all patients
for 10-year mortality prediction. mean (95% CI)

| | Easy | Hard | All |
|---|---|---|---|
| AUC | 0.87 (0.86, 0.89) | 0.46 (0.38, 0.54) | 0.86 (0.84, 0.98) |
| Sens | 0.81 (0.79, 0.82) | 0.62 (0.55, 0.69) | 0.79 (0.77, 0.81) |
| Spec | 0.84 (0.81, 0.86) | 0.29 (0.17, 0.41) | 0.79 (0.76, 0.82) |
| PPV | 0.94 (0.92, 0.95) | 0.72 (0.65, 0.79) | 0.92 (0.90, 0.93) |
| NPV | 0.59 (0.56, 0.63) | 0.20 (0.12, 0.30) | 0.56 (0.53, 0.59) |
| Acc | 0.81 (0.80, 0.83) | 0.54 (0.48, 0.60) | 0.79 (0.77, 0.80) |

An example embodiment of an FSA-based patient grouping method is an intuitive method with a clear clinical meaning associated with the FFC prediction. The grouping method identifies the hard-to-predict group that having much lower performance. This may indicate that additional clinical variables are needed to improve the discriminative power for the hard-to-predict group patients.

### DISCUSSION

Early and timely diagnosis and treatment remains one of the major challenges in the healthcare industry. Use of AI models has repeatedly proven its strong potential in early diagnosis, detection, and treatment in various healthcare applications. However, these AI models require a large amount of feature input, which could potentially delay the decision due to the data gathering and unavoidably increase the cost of healthcare. It was hypothesized that not every patient requires all features to make confident prediction. An example embodiment of a Bayesian-based computational framework was developed to quantitatively identify the necessity of features for making confident prediction in a personalized manner.

An example embodiment of a system, referenced to interchangeably herein as a Feature Sufficiency Analysis (FSA) system or computer-based system, may be a simple, model-agnostic, Bayesian-based, and personalized computational framework designed to estimate the feature sufficiency for confident prediction. To define the confident prediction, a full-feature-capacity (FFC) prediction was introduced, which refers to ML prediction with all features. A confident prediction for a patient with only a subset of feature available means that, regardless of the values of missing features, the ML prediction will remain the same. Thus, a patient reaching the FFC prediction may be referred to as an FFC-predictable patient. For prolonged ventilation prediction, 86% patients only need a half of features to reach FFC prediction. 75% patients reach FFC prediction with less than half of time and monetary cost. For 10-year mortality prediction, 91% patients reach FFC prediction with a half of features. This system shows a strong potential to reduce the time and monetary cost for applying the healthcare ML model. Additionally, leveraging the FSA system and the goal of reaching FFC prediction, an example embodiment of an FSA-based feature ranking method and a patient grouping method was developed to identify hard-to-predict patients. Both methods are developed based on the minimum number of necessary features for FFC prediction.

An example embodiment of a system disclosed herein provides a quantitative recommendation for one of the most common daily jobs of clinicians: whether it is necessary to perform an extra test or procedure of their patients, or the available (provided, obtained) variables are enough. The system is principled, highly intuitive and explainable such that the derived tools including feature ranking and patient grouping can be easily understood. This system is a model-agnostic framework such that all healthcare machine learning models, even non-ML models, can be applied. Finally, it was shown that for cases in which prediction of patients is made regardless of whether they are reaching FFC prediction, the FSA-based risk score has a better performance than the sub-model method.

Identifying hard-to-predict group patients for whom the model is less predictive is useful for the use in the clinical settings. A major effort in ML in healthcare is to identify the predictive uncertainty (Chua, M. et al. Tackling prediction uncertainty in machine learning for healthcare. Nat Biomed Eng 7, 711-718 (2023)). Predictive uncertainty (PU) is the distribution associated with the risk score for each patient (Tyralis, H. & Papacharalampous, G. A review of predictive uncertainty estimation with machine learning. Artif Intell Rev 57, (2024)). We would like to test if patients with high PU is correlated with our hard-to-predict group. We applied the forestCI (Wager, S., Hastie, T. & Efron, B. Confidence intervals for random forests: The jackknife and the infinitesimal jackknife. The journal of machine learning research 15, 1625-1651 (2014)), a widely used predictive uncertainty estimator for random forest, to obtain the predictive uncertainty. For each prediction tasks, an example embodiment may take the top n patients with the highest PU. The n matches the number of patients in a hard-to-predict group.

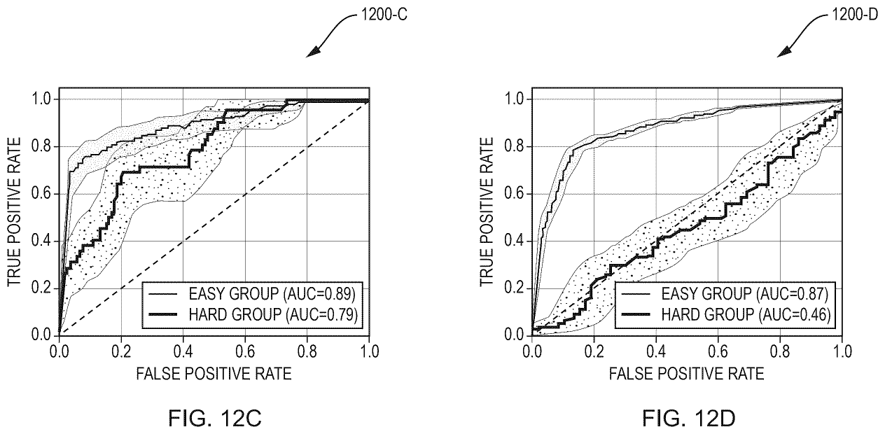

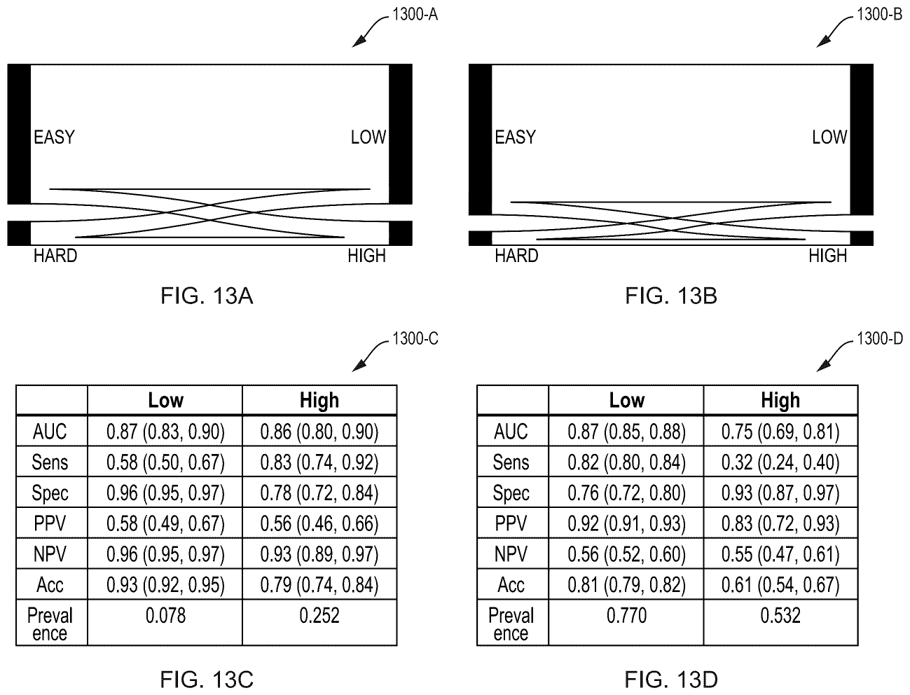

FIGS. 13A and 13B are graphs (1300-A, 1300-B) that show that a hard-to-predict group based on an example embodiment of system disclosed herein does not overlap strongly with a high predictive uncertainty (PU) group.

FIGS. 13C and 13D are tables (1300-C, 1300-D) that demonstrate that the high PU groups have an AUC 0.86 (0.80-0.90) for prolonged ventilation prediction and 0.75 (0.69-0.81) for 10-year mortality prediction, both has higher performance than the hard-to-predict group, AUC 0.79 (0.71, 0.86) for prolonged ventilation prediction and AUC 0.46 (0.38, 0.54) for 10-year mortality prediction. This shows that an example embodiment of a method disclosed herein is better in identifying the low performance group.

Two future research directions of this work are proposed. 1. For a patient with only a portion of features available and does not reach FFC prediction, is it possible to identify which feature to measure such that the patient has the largest likelihood to reach FFC prediction? A relevant ML research field is the dynamic feature selection. Multiple methods have been developed including using reinforcement or greedy selection (Covert, I. C., Qiu, W., Lu, M. & Kim, N. Y. Learning to maximize mutual information for dynamic feature selection. International (2023)). Dynamic feature selection methods can be explored based on an example embodiment of an FSA system disclosed herein. 2. Determine impact of time on the FFC prediction. Different features are typically measured at different time points. While waiting for additional features, the measured features such as lab results and vital signs will also change, thus, putting a source of uncertainty. This source of uncertainty can influence the prediction and may potentially drag the prediction from FFC prediction.

In summary, an example embodiment of an FSA system has demonstrated a strong capacity to improve the healthcare ML model in practice, an ML models in general. It is a principled, easy-to-use system such that can be easily applied to existed clinical score models or an any other ML model. The FSA system can significantly reduce the time and monetary cost for feature collection, while preserving the model performance. The FSA system may be used as a life-saving tool in a clinical setting.

### Methods

### Datasets

STS Heart surgery cohort One heart surgery patient cohort was obtained by querying the STS Adult Cardiac Surgery Database (ACSD) (Bowdish, M. E. et al. STS Adult Cardiac Surgery Database: 2021 update on outcomes, quality, and research. Ann. Thorac. Surg. 111, 1770-1780 (2021)) to develop a dataset including cardiac surgery cases from the Maine Medical Center over a 10-year period from Jan. 1, 2012 to Dec. 31, 2021. Data were harmonized as there were various iterations of the STS ACSD. All patient identifiers and private health information (PHI) were removed for patient protection. The project was submitted to the Maine Medical Center (MMC) Institutional Review Board (IRB), who determined the project to be “non-research” in a letter dated Sep. 11, 2021.

NHANES cohort National Health and Nutrition Examination Survey (NHANES) is a publicly-available US national outpatient cohort of 13,442 patients and 35 features (Miller, H. W. Plan and operation of the health and nutrition examination survey. United states—1971-1973. Vital Health Stat. 1 1-46 (1973)) from 1971 to 1974. The 10-year mortality status was followed up in 1992.

The time cost for STS features were assessed by two clinical practitioners at Maine Health center and presented in Table 600-C of FIG. 6C, disclosed above. Variables are grouped based on the Test/Action it requires to obtain. For each Test/Action, the time cost is provided in a range. In this study, the minimum value of the range was used to represent the time cost. The monetary cost for STS variables is obtained from Hospital Price Index found at search.hospitalpriceindex.com/hpi2/machineReadable/mainemedicalcenter/7975or). Note that one test can generate multiple variables. For example, Echo ultrasound provides both vdinsufm and hdef. Thus, cost analysis on the ML model in FIGS. 9A and 9B is based on Test/Action rather than variables. Erion et al. (Erion, G. et al. A cost-aware framework for the development of AI models for healthcare applications. Nat Biomed Eng 6, 1384-1398 (2022)) provided the monetary cost for the 35 features in NHANES dataset. They assigned costs to features by referencing the Medicare data for lab tests (Clinical Laboratory Fee Schedule Files-Cy 2019 Q3 Release (Centers for Medicare and Medicaid Services, 2019); cms.gov/Medicare/Medicare-Fee-for-Service-Payment/ClinicalLabFeeSched/Clinical-Laboratory-Fee-Schedule-Files.html). All other variables are considered monetary cost.

### Baseline ML Model

The raw data are imputed by the mean value for the continuous variable and mode value for the categorical variables. The data is stratified randomly sampled into training (70% for STS dataset and 60% for NHANES dataset), validation (10% for STS dataset and 20% for NHANES dataset) and test (20%) set.

The aim of the ML model for the heart surgery data is to predict the occurrence of postoperative prolonged ventilation. To do this, a collection of the 72 O'Brien expert-selected features (O'Brien, S. M. et al. The Society of Thoracic Surgeons 2018 Adult Cardiac Surgery Risk Models: Part 2-Statistical Methods and Results. Ann. Thorac. Surg. 105, 1419-1428 (2018)) was used to predict the prolonged ventilation. A random forest model was developed on 72 features with 0.89 AUC. It was found that using the most important 12 features identified by the feature ranking from the mean decrease of impurity (Breiman, L. Random Forests. Mach. Learn. 45, 5-32 (2001)). The 12-feature model yields 0.88 AUC, 0.67 sensitivity, 0.94 specificity, 0.94 positive predictive value (PPV) and the threshold is 0.285. The baseline ML model was chosen as the 12-feature random forest model for the prediction of the prolonged ventilation for non-limiting example. 12 features are shown in the Table 1, above.

The task for NHANES dataset is to develop a ML model to predict 10-year mortality with 35 features. A random forest model was also developed on 35 features with 0.86 AUC, 0.79 sensitivity, 0.79 specificity, 0.92 positive predictive value (PPV), 0.56 negative predictive value (NPV). The threshold of the risk score is 0.696.

### Feature Sufficiency Analysis (FSA) System

According to an example embodiment, a Feature Sufficiency Analysis (FSA) system disclosed herein may be a Bayesian-based computational framework. The system aims to identify the effect of missing variables on the binary prediction. Using a baseline ML model, a prediction made with a complete set of features is defined as the full-feature capacity (FFC) prediction. For a patient with only a subset of features available, if the prediction remains the same regardless of the values of the rest of missing variables, the subset of features for this patient may be considered sufficient, reaching the full-feature capacity (FFC) prediction. This indicates that the decision made with the available (provided) subset of features remains unchanged even when considering all possible values for the missing features. Namely, missing variables do not affect the binary prediction.

### The FSA System Analyzed the Impact of Missing Values on Prediction Through a Three-Step Process.

1. Posterior distributions for missing values. For each patient, a Monte Carlo approximation was performed to estimate the posterior distributions for missing values conditional on the available features, represented as P (missing variables|available variables). Multiple Imputation was applied with chained equations, a widely-used numerical method to estimate the posterior distribution for missing values. This is a distribution-free Monte Carlo method for both numerical and categorical variables. For this study, 100 Monte Carlo realizations were generated to approximate these posterior distributions.
2. Risk score calculation. Each of the 100 Monte Carlo realizations, combined with the available feature values was put into the baseline ML model to obtain the corresponding risk scores and, thus, 100 risk scores were obtained from 100 realizations in step 1.
3. Risk score distribution analysis. 100 risk scores form a risk score distribution driven by the posterior distributions of missing values. By examining the relative position between risk score distribution and the threshold of an ML model, an effect of missing values on prediction could be assessed. If all 100 risk scores exceed the threshold, it indicates that, regardless of the missing values, the ML model will make a positive prediction, and vice versa. One may consider this patient with a subset of features available reaching the FCC prediction. If some risk scores are above and some are below the threshold, it indicates that missing variables can affect the prediction. In other words, the risk score distribution intersects with the threshold. One may consider that this patient requires missing variables to reach FCC prediction.

### Feature Ranking Based on FSA System

The ablation study was performed to evaluate the feature ranking based on the FSA system. Ablation study essentially assesses the feature importance by evaluating how removing/ablating this feature will affect the model performance. In this study, the feature to be assessed was removed from the test set and set as missing. Then, the feature importance was ranked by the number of patients for whom FCC prediction cannot be achieved with this specific feature being removed. A greater number of patients indicates a greater importance for this feature.

Comparison with Other Feature Ranking Methods

An example embodiment of a FSA feature ranking disclosed herein was compared with 4 widely used feature ranking methods in the ML community: mean decrease of impurity (Breiman, L. Random Forests. Mach. Learn. 45, 5-32 (2001)), permutation (Breiman, L. Random Forests. Mach. Learn. 45, 5-32 (2001)), logistic regression and SHAP (Lundberg, S. M. & Lee, S. I. A unified approach to interpreting model predictions. Adv. Neural Inf. Process. Syst. (2017)). The Spearman correlation was performed to show the pairwise correlation between different feature ranking methods.

### Validation for the Posterior Distribution of Missing Variables.

A faithful estimation of the posterior distribution for missing values is a useful action for the FSA system. The posterior distribution was validated by leveraging the posterior distribution from the ablation study for feature ranking. For continuous variables, a credible interval was set on the posterior distribution for all patients in the test set, centered at 50%. For example, a 90% credible interval ranges from 5% to 95% of the posterior distribution. Then the number of patients whose actual variable values fall within this credible interval range was counted. The percentage of patients falling within the range should match the credible interval range. To validate the posterior distribution, the credible interval range was varied from 1% to 99% in 1% increments and the corresponding percentages of patients falling within the credible interval was calculated. For the categorical variables, the calibration plot was performed on the test set data for each unique value of the variable. The calibration plot shows the predicted probability of a value for the categorical variable from the posterior distribution and the percentage of patients having that specific value.

### Cumulative Feature Sufficiency Analysis

With a machine learning predictor with K features, an order of the feature was defined first. The order can be based on the feature importance or the time/monetary cost of features. Then, the first n features available (provided) was made based on the order and the FSA system was used to identify the risk score distribution for the first n features. Then n was enumerated from 1 to K. For one patient, it enables analysis of the evolution of the risk score with the increase of n and the minimum necessary number of features to reach FFC prediction given the feature order to be identified. For a patient cohort, the percentage of patients who can reach FFC prediction with first n features can be identified.

### Cost Analysis

In the NHANES cohort, each feature assigns a monetary cost. For the STS heart surgery cohort, features are grouped based on the clinical test/actions to share the time and monetary cost. Similar to cumulative feature sufficiency analysis, an order from low to high cost was formed and the percentage of FFC-predictable patients with first n features based on the order of cost were identified. For the STS heart surgery cohort, since features are grouped based on clinical test/actions, rather than first n features, the first n clinical test/actions were used to perform the analysis.

ML Performance with Missing Variables

An example embodiment of an FSA system disclosed herein is able to provide a risk score distribution for patients with missing variables. The mean of the risk score distribution was taken from FSA to obtain the prediction by comparing the mean risk score with the threshold. For comparison, another strategy for dealing with missing values is to develop a collection of sub-models, where each model is based on a unique subset of features (Erion, G. et al. A cost-aware framework for the development of AI models for healthcare applications. Nat Biomed Eng 6, 1384-1398 (2022)). Thus, prediction with missing variables only needs to identify the right sub-model that is trained with available features.

Based on the feature importance order, the first n features were made available and the ML performance evaluated by AUC. A value for n was enumerated from 1 to K to analyze how AUC evolves with more features being available for both FSA system method and sub-model method.

### Patient Grouping Based on the FSA System.

Following the feature importance order, the cumulative feature sufficiency analysis was performed to identify the minimum number of features necessary to reach FFC prediction for every patient. Patients that require less or equal to half of the total number of features to reach FFC prediction were defined to as an “easy group,” and those who require more than half of total number to reach FFC prediction as a “hard group”. The two groups of patients were compared by evaluating the ML performance on both groups.

FIG. 14 is a block diagram of an example of the internal structure of a computer 1400 in which various embodiments of the present disclosure may be implemented. The computer 1400 contains a system bus 1402, where a bus is a set of hardware lines used for data transfer among the components of a computer or digital processing system. The system bus 1402 is essentially a shared conduit that connects different elements of a computer system (e.g., processor, disk storage, memory, input/output ports, network ports, etc.) that enables the transfer of information between the elements. Coupled to the system bus 1402 is an I/O device interface 1404 for connecting various input and output devices (e.g., keyboard, mouse, display monitors, printers, speakers, microphone, etc.) to the computer 1400. A network interface 1406 allows the computer 1400 to connect to various other devices attached to a network (e.g., global computer network, wide area network, local area network, etc.). Memory 1408 provides volatile or non-volatile storage for computer software instructions 1410 and data 1412 that may be used to implement embodiments (e.g., method 200) of the present disclosure, where the volatile and non-volatile memories are examples of non-transitory media. Disk storage 1413 also provides non-volatile storage for the computer software instructions 1410 and data 1412 that may be used to implement embodiments (e.g., method 200) of the present disclosure. A central processor unit 1418 is also coupled to the system bus 1402 and provides for the execution of computer instructions.

Further example embodiments disclosed herein may be configured using a computer program product; for example, controls may be programmed in software for implementing example embodiments. Further example embodiments may include a non-transitory computer-readable-medium that contains instructions that may be executed by a processor, and, when loaded and executed, cause the processor to complete methods and techniques described herein. It should be understood that elements of the block and flow diagrams may be implemented in software or hardware, such as via one or more arrangements of circuitry of FIG. 22, disclosed above, or equivalents thereof, firmware, a combination thereof, or other similar implementation determined in the future.

In addition, the elements of the block and flow diagrams described herein may be combined or divided in any manner in software, hardware, or firmware. If implemented in software, the software may be written in any language that can support the example embodiments disclosed herein. The software may be stored in any form of computer readable medium, such as random-access memory (RAM), read only memory (ROM), compact disk read-only memory (CD-ROM), and so forth. In operation, a general purpose or application-specific processor or processing core loads and executes software in a manner well understood in the art. It should be understood further that the block and flow diagrams may include more or fewer elements, be arranged or oriented differently, or be represented differently. It should be understood that implementation may dictate the block, flow, and/or network diagrams and the number of block and flow diagrams illustrating the execution of embodiments disclosed herein.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A computer-implemented method for feature-based machine learning (ML) model prediction, the computer-implemented method comprising:
using an imputation method to produce posterior distributions of unprovided features of a set of retrospective features, the posterior distributions produced based on the set of retrospective features and provided features of the set of retrospective features;
producing, by an ML model, a threshold and a risk score distribution of a prediction of an event, the producing based on the posterior distributions produced by the imputation method used and the provided features, the ML model trained on the set of retrospective features;
selecting at least one unprovided feature from a partial set of the unprovided features to improve predictive accuracy of the ML model iteratively, the selecting based on the threshold and the risk score distribution of the prediction of the event; and
outputting a representation of the at least one unprovided feature selected toward approximating a full-feature-capacity (FFC) prediction with a partial set of the retrospective features, the FFC prediction based on the set of retrospective features in its entirety, the representation output causing the at least one unprovided feature to be provided for a subsequent iteration, the partial set of the retrospective features including the provided features supplemented by the at least one unprovided feature selected and provided at the subsequent iteration.

2. The computer-implemented method of claim 1, wherein the event is at least one event of a plurality of events and wherein the computer-implemented method further comprises:
applying at least one criterion to reduce the partial set of the unprovided features as a function of at least one characterization of the at least one event, and wherein a given event of the at least one event is a time sensitive event.

3. The computer-implemented method of claim 1, further comprising:
determining, based on the threshold and the risk score distribution, whether the provided features are sufficient for the ML model to approximate the FFC prediction;
performing the selecting of the at least one unprovided feature and the outputting of the representation responsive to determining that the provided features are not sufficient for approximating the FFC prediction; and
outputting the prediction responsive to determining that the provided features are sufficient for approximating the FFC prediction.

4. The computer-implemented method of claim 1, wherein the event is a medical event for a patient and wherein the computer-implemented method further comprises producing a decision based on the prediction output, wherein the decision produced influences triage of the patient to prevent the medical event.

5. The computer-implemented method of claim 1, wherein the set of retrospective features includes clinical features of patients on a per-patient basis, wherein the event is associated with a medical outcome of a patient, wherein the risk score distribution of the prediction represents certainty of the prediction in a presence of the unprovided features, and wherein the threshold is learned by the ML model from the set of retrospective features in a training phase of the ML model.

6. The computer-implemented method of claim 1, wherein the representation indicates a respective feature importance ranking for each unprovided feature selected of the at least one unprovided feature selected and wherein the respective feature importance ranking indicates relative importance, among the at least one unprovided feature selected, toward improving the predictive accuracy of the ML model.

7. The computer-implemented method of claim 1, wherein the using, producing, selecting, and outputting are performed in a current iteration and wherein the computer-implemented method further comprises:
acquiring the at least one unprovided feature selected, the acquiring responsive to the outputting of the current iteration; and
updating the provided features to include the at least one unprovided feature selected and acquired for use in the subsequent iteration.

8. The computer-implemented method of claim 7, wherein the acquiring includes causing at least one device to perform at least one measurement to measure an unprovided feature of the at least one unprovided feature selected.

9. The computer-implemented method of claim 1, further comprising employing the computer-implemented method in a computer-based tool for clinical evaluation of a patient and performing, by the computer-based tool, dynamic risk assessment of the patient based on the threshold and the risk score distribution of the prediction of the event, wherein the event is a medical outcome for the patient.

10. The computer-implemented method of claim 1, wherein the event is a medical outcome for a patient and wherein the computer-implemented method further comprises outputting an indication that represents at least one actionable component for preventing the medical outcome from occurring.

11. The computer-implemented method of claim 1, wherein the ML model is a supervised ML model.

12. A computer-based system for feature-based machine learning (ML) model prediction, the computer-based system comprising:
at least one processor; and
at least one memory, the at least one having encoded thereon a sequence of instructions which, when loaded and executed by the at least one processor, causes the computer-based system to:
use an imputation method to produce posterior distributions of unprovided features of a set of retrospective features, the posterior distributions produced based on the set of retrospective features and provided features of the set of retrospective features;
employ an ML model to produce a threshold and a risk score distribution of a prediction of an event, the producing based on the posterior distributions produced by the imputation method used and the provided features, the ML model trained on the set of retrospective features;
select at least one unprovided feature from a partial set of the unprovided features to improve predictive accuracy of the ML model iteratively, selection of the at least one unprovided feature being based on the threshold and the risk score distribution of the prediction of the event; and
output a representation of the at least one unprovided feature selected toward approximating a full-feature-capacity (FFC) prediction with a partial set of the retrospective features, the FFC prediction based on the set of retrospective features in its entirety, the representation output causing the at least one unprovided feature to be provided for a subsequent iteration, the partial set of the retrospective features including the provided features supplemented by the at least one unprovided feature selected and provided at the subsequent iteration.

13. The computer-based system of claim 12, wherein the event is at least one event of a plurality of events and wherein the sequence of instructions, when loaded and executed by the at least one processor, further causes the computer-based system to:
apply at least one criterion to reduce the partial set of the unprovided features as a function of at least one characterization of the at least one event, and wherein a given event of the at least one event is a time sensitive event.

14. The computer-based system of claim 12, wherein the event is a medical event for a patient, and wherein the sequence of instructions, when loaded and executed by the at least one processor, further causes the computer-based system to:
determine, based on the threshold and the risk score distribution, whether the provided features are sufficient for the ML model to approximate the FFC prediction;
perform selection of the at least one unprovided feature and output of the representation responsive to determining that the provided features are not sufficient for approximating the FFC prediction;
output the prediction responsive to determining that the provided features are sufficient for approximating the FFC prediction; and
produce a decision based on the prediction output, the decision produced influences triage of the patient.

15. The computer-based system of claim 12, wherein the set of retrospective features includes clinical features of patients on a per-patient basis, wherein the event is associated with a medical outcome of a patient, wherein the risk score distribution of the prediction represents certainty of the prediction in a presence of the unprovided features, and wherein the threshold is learned by the ML model from the set of retrospective features in a training phase of the ML model.

16. The computer-based system of claim 12, wherein the representation indicates a respective feature importance ranking for each unprovided feature selected of the at least one unprovided feature selected and wherein the respective feature importance ranking indicates relative importance, among the at least one unprovided feature selected, toward improving the predictive accuracy of the ML model.

17. The computer-based system of claim 12, wherein the sequence of instructions, when loaded and executed by the at least one processor, further causes the computer-based system to:
acquire the at least one unprovided feature selected responsive to outputting the representation in a current iteration; and
update the provided features to include the at least one unprovided feature selected and acquired for use in the subsequent iteration, wherein acquiring the at least one unprovided feature selected includes causing at least one device to perform at least one measurement to measure an unprovided feature of the at least one unprovided feature selected.

18. The computer-based system of claim 12, wherein the computer-based system is a tool for clinical evaluation of a patient and wherein the sequence of instructions, when loaded and executed by the at least one processor, further causes the computer-based system to:
perform dynamic risk assessment of the patient based on the threshold and the risk score distribution of the prediction of the event, wherein the event is a medical outcome for the patient; and
output an indication that represents at least one actionable component for preventing the medical outcome from occurring.

19. The computer-based system of claim 12, wherein the ML model is a supervised ML model.

20. A non-transitory computer-readable medium having encoded thereon a sequence of instructions which, when loaded and executed by at least one processor, causes the at least one processor to:
use an imputation method to produce posterior distributions of unprovided features of a set of retrospective features, the posterior distributions produced based on the set of retrospective features and provided features of the set of retrospective features;
employ an ML model to produce a threshold and a risk score distribution of a prediction of an event, the producing based on the posterior distributions produced by the imputation method used and the provided features, the ML model trained on the set of retrospective features;
select at least one unprovided feature from a partial set of the unprovided features to improve predictive accuracy of the ML model iteratively, selection of the at least one unprovided feature being based on the threshold and the risk score distribution of the prediction of the event; and
output a representation of the at least one unprovided feature selected toward approximating a full-feature-capacity (FFC) prediction with a partial set of the retrospective features, the FFC prediction based on the set of retrospective features in its entirety, the representation output causing the at least one unprovided feature to be provided for a subsequent iteration, the partial set of the retrospective features including the provided features supplemented by the at least one unprovided feature selected and provided at the subsequent iteration.
